(19) United States
(12) Patent Application Publication
(10) Pub. No.: US 2024/0285401 A1
(43) Pub. Date: Aug. 29, 2024

(54) PROSTHETIC HEART VALVES WITH RELEASABLY ATTACHED OUTER SKIRTS AND METHODS ASSOCIATED THEREWITH

(71) Applicant: EDWARDS LIFESCIENCES CORPORATION, Irvine, CA (US)

(72) Inventors: Tamir S. Levi, Zikhron Yaakov (IL); Elena Sherman, Pardes Hana (IL); Melissa Fusari, Boise, ID (US); Nikolai Gurovich, Hadera (IL)

(21) Appl. No.: 18/658,051

(22) Filed: May 8, 2024

Related U.S. Application Data
(63) Continuation of application No. PCT/US2022/049661, filed on Nov. 11, 2022.
(60) Provisional application No. 63/401,981, filed on Aug. 29, 2022.
(60) Provisional application No. 63/279,095, filed on Nov. 13, 2021.

(51) Int. Cl. A61F 2/24

(52) U.S. Cl. CPC A61F 2/2418; A61F 2/2412; A61F 2/2433; A61F 2210/0057; A61F 2220/005; A61F 2220/0075; A61F 2250/0004; A61F 2250/0039; A61F 2250/006; A61F 2250/0097; A61F 2250/0098

(57) ABSTRACT

A prosthetic heart valve including an annular frame and one or more detachable outer skirts and methods associated therewith. The frame has one or more outer skirts releasbly attached to an exterior surface of the frame. In some examples, an outer skirt is attached to the frame via at least one retaining suture formed by stitches looped around a pull suture and struts of the frame. Portions of the outer skirt are captured between the pull suture and the exterior surface of the frame along a suture line. Removal of the pull suture from the suture line enables the outer skirt and frame to separate as the stitches are pulled through openings in the outer skirt. In some examples, outer skirts can be releasably attached to the frame via bioresorbable couplers. Removability of the outer skirts can facilitate improvements in implant and explant procedures of the prosthetic valves.

### CROSS REFERENCE TO RELATED APPLICATIONS

This application is a continuation of PCT Application No. PCT/US2022/049661, filed Nov. 11, 2022, which claims the benefit of U.S. Provisional Patent Application Nos. 63/279,095, filed on Nov. 13, 2021, and 63/401,981, filed on Aug. 29, 2022, each of which is incorporated herein by reference.

### FIELD

The present disclosure relates to prosthetic heart valves, including frames and an attached valve structure and a releasably attached outer skirt, as well as methods of manufacture and methods of implantation and explant thereof.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting and explanting of these devices and valves in humans. Various surgical approaches (such as, for example, percutaneous and minimally-invasive procedures or open heart procedures) are used to deliver and/or remove prosthetic medical devices to locations inside the body.

In one specific example of a minimally invasive implantation procedure, a prosthetic heart valve can be mounted in a crimped state on the distal end of a delivery device and advanced through the patient's vasculature (for example, through a femoral artery and the aorta) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery device so that the prosthetic valve can self-expand to its functional size. The prosthetic valve is then operational inside of the patient's heart and replaces the function of the native heart valve for the useful life of the prosthetic valve, which typically is about 15 to 25 years.

Most expandable, transcatheter heart valves comprise a radially expandable and compressible cylindrical metal frame and prosthetic leaflets mounted inside the frame. The frame can comprise a plurality of circumferentially extending rows of angled struts defining rows of open cells of the frame. The prosthetic valve can include an outer sealing member (also referred to as an “outer skirt”) affixed to an outer surface of the frame for sealing the prosthetic valve against tissue of the native heart valve. The outer skirt typically is attached to frame via sutures. The outer skirt typically encourages tissue ingrowth, which helps seal and anchor the prosthetic valve against the native annulus. The use of an outer skirt is particularly important for patients suffering from aortic stenosis (AS), which causes calcification of the native aortic valve. The calcification can create an irregularly shaped surface on the native annulus tissue. Outer skirts have been designed to fill gaps between the frame of the prosthetic valve and the hardened calcium deposits of the surrounding tissue to establish a seal between the prosthetic valve and the native annulus.

In some patients, it may become desirable to remove or explant the prosthetic valve if the valve malfunctions, experiences wear and tear or deterioration from use over time, and/or as advances in prosthetic implant technologies are developed and an improved prosthetic valve device is desired. During explant procedures, the older prosthetic valve is removed such that a new prosthetic heart valve can be implanted within the diseased native valve. However, explantation of conventional prosthetic valves can be quite challenging due to the neointimal tissue formed between the outer skirt and the native annulus. Typically, the surgeon is required to cut the tissue surrounding the prosthetic valve in order to remove the prosthetic valve from the patient. The outer skirt typically is bounded on one side by the frame of the prosthetic valve and on other side by the native annulus, leaving little room for the surgeon to access the tissue that needs to be cut from the prosthetic valve. As can be appreciated, this is a delicate procedure and may involve significant risks to the patient. In some cases, for example, explantation of a prosthetic valve can result in trauma to the tissue of the left ventricular outflow tract.

Further, in some patients, such as patients suffering from aortic insufficiency (AI), the native valve may be dilated and require implantation of an oversized prosthetic valve in order to create sufficient contact for sealing between the native annulus tissue and the prosthetic valve implanted therein. Compared to a stenosed native valve, the tissue of an insufficient native valve is more pliable and can more easily conform to the frame of the prosthetic for establishing a seal between the prosthetic valve and the native annulus. Thus, in some cases, an outer skirt may not be required, but unlike a stenosed native valve, it is much more difficult to anchor the prosthetic valve in place due to the pliable nature of the native tissue. As such, anchoring features, such as projections on an exterior surface of a frame of the prosthetic valve, can assist in maintaining a position of the implanted prosthetic valve. However, use of an oversized valve for AI patients may hinder or complicate transcatheter delivery, particularly if the patient's vasculature is small and/or if the oversized valve includes an outer skirt, which contributes to a larger crimp profile of the prosthetic valve during transcatheter delivery thereof. Further, the outer skirt may cover or block some or all of the anchoring features on the prosthetic valve when implanted and prevent anchoring. Thus, a prosthetic valve primarily intended for implantation within a stenosed native valve may not be ideal for implantation with an insufficient native valve, and vice versa.

### SUMMARY

Described herein are prosthetic heart valves, delivery apparatus, and methods for implanting prosthetic heart valves. The disclosed prosthetic heart valves, delivery apparatus, and methods can, for example, provide an outer skirt that is releaseably attached to a frame of the prosthetic valve via the at least one retaining suture and the one or more pull sutures. As such, the devices and methods disclosed herein can, among other things, overcome one or more of the deficiencies of typical prosthetic heart valves and their associated implant and/or explant procedures.

A prosthetic heart valve can comprise a frame and a valve structure coupled to the frame. In addition to these components, a prosthetic heart valve can further comprise one or more of the components disclosed herein, such as, for example, a detachable outer skirt. In some examples, the detachable outer skirt can be attached to the frame via at least one retaining suture and one or more pull sutures arranged along a suture line.

In some examples, a prosthetic heart valve comprises an annular frame comprising a plurality of interconnected struts; and an outer skirt disposed on an outer surface of the annular frame and detachably secured to at least a portion of the plurality of interconnected struts. The outer skirt can be detachably secured to the portion of the plurality of interconnected struts via at least one retaining suture and one or more pull sutures, the retaining suture and one or more pull sutures arranged along a suture line, the retaining suture forming a plurality of stitches extending around the portion of the plurality of interconnected struts, through the outer skirt, and over the one or more pull sutures. Further, the outer skirt is configured to detach from the portion of the plurality of interconnected struts and enable separation of the annular frame from the outer skirt via removal of the one or more pull sutures from the suture line and withdrawal of the stitches through the outer skirt.

In some examples, a prosthetic heart valve comprises: a frame comprising a plurality of interconnected struts arranged between an inflow end and an outflow end of the frame; an outer skirt disposed on an exterior surface of the frame; one or more pull sutures disposed on an exterior surface of the outer skirt along a suture line; and at least one retaining suture forming a plurality of stitches looped around the one or more pull sutures and specified ones of the plurality of interconnected struts disposed along the suture line. In such an example, portions of the outer skirt are captured between the pull suture and the exterior surface of the frame along the suture line. Further, the outer skirt is releaseably attached to the frame via the at least one retaining suture and the one or more pull sutures. Yet further, wherein the outer skirt is configured to be released from the frame via removal of the one or more pull sutures from the plurality of stitches along the suture line, and withdrawal of the plurality of stitches through the outer skirt along the suture line. Further still, the plurality of stitches are configured to remain looped around the specified ones of the plurality of interconnected struts along the suture line after the release of the outer skirt from the frame.

In some examples, a method of implanting a prosthetic heart valve includes a plurality of steps comprising: evaluating a heart condition of a patient and size of a native annulus of the patient; based on the heart condition of the patient and size of the native annulus, selecting a prosthetic heart valve having an appropriate size for implantation within the patient, the prosthetic heart valve comprising a frame having an outer skirt detachably secured on an exterior surface thereof, the outer skirt detachably secured to the frame via at least one pull suture and at least one retaining suture forming a plurality of stitches looped around the pull suture and further looped around one or more struts of the frame; if the heart condition of the patient is aortic insufficiency, removing the outer skirt from the frame by grasping an end of the pull suture, and applying a force to the end to withdraw the pull suture from the stitches, and separating the frame from one or more portions of the outer skirt; and implanting the prosthetic heart valve within the native annulus of the patient.

In some examples, a prosthetic heart valve comprises an annular frame comprising a plurality of interconnected struts and a plurality of anchoring projections; an outer skirt disposed on an outer surface of the annular frame and covering at least a portion of the plurality of projections, the outer skirt detachably secured to at least a portion of the plurality of interconnected struts; wherein the outer skirt is detachably secured to the portion of the plurality of interconnected struts via at least one retaining suture and one or more pull sutures, the retaining suture and one or more pull sutures arranged along a suture line, the retaining suture forming a plurality of stitches extending around the portion of the plurality of interconnected struts, through the outer skirt, and over the one or more pull sutures; and wherein the outer skirt is configured to detach from the portion of the plurality of interconnected struts and enable separation of the annular frame from the outer skirt via removal of the one or more pull sutures from the suture line and withdrawal of the plurality of stitches through the outer skirt, the plurality of projections on the outer surface of the annular frame being exposed after separation of the frame and the outer skirt.

In some examples, a method of implanting a prosthetic heart valve, includes a plurality of steps comprising: evaluating a heart condition of a patient and size of a native annulus of the patient; based on the heart condition of the patient and size of the native annulus, selecting a prosthetic heart valve having an appropriate size for implantation within the patient, the prosthetic heart valve comprising a frame having an outer skirt secured on an exterior surface thereof; if the heart condition of the patient is aortic insufficiency, removing the outer skirt from the frame; and implanting the prosthetic heart valve within the native annulus of the patient.

In some examples, a method of explanting a prosthetic heart valve includes a plurality of steps comprising: accessing an implantation site of the prosthetic heart valve, the prosthetic heart valve comprising a frame having an outer skirt detachably secured on an exterior surface thereof, the outer skirt detachably secured to the annular frame via at least one pull suture and at least one retaining suture forming a plurality of stitches looped around the pull suture and further looped around one or more struts of the frame; grasping an end of the pull suture, and applying a force to the end to withdraw the pull suture from the stitches; separating the frame from one or more portions of the outer skirt; and removing the frame from the implantation site.

In some examples, a method of manufacturing a prosthetic heart valve having a detachable outer skirt, the method comprising: obtaining a frame comprising a plurality of interconnected struts arranged between an inflow end and an outflow end of the frame; overlaying one or more portions of the detachable outer skirt on an exterior surface of the frame; and forming a plurality of stitches around selected ones of the plurality of interconnected struts, through the detachable outer skirt, and over one or more pull sutures on an exterior surface of the detachable outer skirt.

In some examples, a method of implanting a prosthetic heart valve, includes a plurality of steps comprising: evaluating a heart condition of a patient and size of a native annulus of the patient; based on the heart condition of the patient and size of the native annulus, selecting a prosthetic heart valve having an appropriate size for implantation within the patient, the prosthetic heart valve comprising a frame having an outer skirt secured on an exterior surface thereof. In some examples, if the heart condition of the patient is aortic insufficiency, removing the outer skirt from the frame and implanting the prosthetic heart valve within the native annulus of the patient without the outer skirt. In some examples, if the heart condition of the patient is aortic stenosis, implanting the prosthetic heart valve within the native annulus of the patient with the outer skirt.

In some examples, a prosthetic heart valve comprises: an annular frame comprising a plurality of interconnected struts and a plurality of anchoring projections; and an outer skirt disposed on an outer surface of the annular frame and covering at least a portion of the plurality of projections, the outer skirt detachably secured to at least a portion of the plurality of interconnected struts. In some examples, the outer skirt is configured to detach from the portion of the plurality of interconnected struts and enable separation of the annular frame from the outer skirt, the plurality of projections on the outer surface of the annular frame being exposed after separation of the frame and the outer skirt.

In some examples, a prosthetic heart valve comprises: an annular frame comprising a plurality of interconnected struts; and an outer skirt disposed on an outer surface of the annular frame and detachably secured to at least a portion of the plurality of interconnected struts, wherein the outer skirt comprises at least one fused edge. In some examples, the fused edge is folded over an exterior surface of the prosthetic heart valve to create a folded edge. In some examples, the outer skirt is detachably secured to the portion of the plurality of interconnected struts along the folded edge via at least one retaining suture and one or more pull sutures, the retaining suture and one or more pull sutures arranged along a suture line, the retaining suture forming a plurality of stitches extending through the outer skirt and over the one or more pull sutures. In some examples, the outer skirt is configured to detach from the portion of the plurality of interconnected struts along the folded edge and enable separation of the annular frame from the outer skirt via removal of the one or more pull sutures from the suture line and withdrawal of the plurality of stitches through the outer skirt.

In some examples, a prosthetic heart valve comprises: an annular frame comprising a plurality of interconnected struts; and an outer skirt disposed on an outer surface of the annular frame and detachably secured to at least a portion of the plurality of interconnected struts. In some examples, the outer skirt is detachably secured to the portion of the plurality of interconnected struts via at least one retaining suture, one or more pull sutures, and a plurality of non-penetrating stitches, the retaining suture, the one or more pull sutures, and the plurality of non-penetrating stitches arranged along a suture line, the retaining suture forming a plurality of stitches extending through the outer skirt and over the one or more pull sutures, the non-penetrating stitches extending around the portion of the plurality of interconnected struts and between the at least one retaining suture and an interior surface of the outer skirt. In some examples, the outer skirt is configured to detach from the portion of the plurality of interconnected struts and enable separation of the annular frame from the outer skirt via removal of the one or more pull sutures from the suture line and withdrawal of the plurality of stitches through the outer skirt.

The above method(s) can be performed on a living animal or on a simulation, such as on a cadaver, cadaver heart, anthropomorphic ghost, simulator (e.g., with body parts, heart, tissue, etc. being simulated).

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a perspective view of a prosthetic heart valve, according to one example.

FIG. 2 is a side view of an example of a delivery apparatus configured to deliver and implant a radially expandable prosthetic heart valve at an implantation site.

FIG. 3 is a perspective view of another example of a prosthetic heart valve.

FIG. 4 is a perspective view of another example of a prosthetic heart valve comprising a frame and an outer skirt secured to the frame.

FIGS. 5A and 5B are perspective views of another example of a prosthetic heart valve comprising a frame and an outer skirt releasably attached to the frame, in accordance with the present disclosure.

FIG. 5C is a top plan view of an example of a portion of the outer skirt for use with the prosthetic heart valve of FIGS. 5A and 5B.

FIG. 5D is an exterior perspective view of another example of a prosthetic heart valve comprising a frame and multiple outer skirt portions attached to the frame, in accordance with the present disclosure.

FIG. 6A is an illustration of an example of a suture line configuration for use with the prosthetic heart valves disclosed herein.

FIG. 6B is a cross-sectional view of the exemplary suture line configuration shown in FIG. 6A taken along line 6B-6B.

FIG. 7A is a logical flow diagram of an exemplary method for implant of a prosthetic heart valve having a detachable outer skirt, in accordance with the present disclosure.

FIG. 7B is a logical flow diagram of an exemplary method for explant of a prosthetic heart valve having a detachable outer skirt, in accordance with the present disclosure.

FIG. 7C is a logical flow diagram of an exemplary method for separation of a detachable outer skirt and a frame of a prosthetic valve, in accordance with the present disclosure.

FIGS. 8A-8D are perspective views of another example prosthetic heart valve having a detachable outer skirt, showing various stages during detachment of the outer skirt from the frame.

FIG. 9A is a perspective view of an exemplary prosthetic heart valve assembly, according to another example.

FIG. 9B is a detail view of the prosthetic heart valve assembly of FIG. 9A.

FIG. 9C is a perspective view of a valve frame of the prosthetic heart valve assembly of FIG. 9A, depicting the valve frame in an annular configuration.

FIG. 9D is a perspective view of an anchoring frame of the prosthetic heart valve assembly of FIG. 9A, depicting the anchoring frame in an annular configuration.

FIG. 9E is a side elevation view of the valve frame of the prosthetic heart valve assembly of FIG. 9A, depicting the valve frame in a flat configuration.

FIG. 9F is a side elevation view of the anchoring frame of the prosthetic heart valve assembly of FIG. 9A, depicting the anchoring frame in a flat configuration.

FIG. 10A is an illustration of another example a prosthetic heart valve comprising a frame and an outer skirt releasably attached to the frame, in accordance with the present disclosure.

FIGS. 10B and 10C are interior perspective views of the outflow end and the inflow end of the prosthetic heart valve of FIG. 10A.

FIG. 11A is an illustration of another example of a suture line configuration for use with the prosthetic heart valves disclosed herein.

FIG. 11B is a cross-sectional view of the exemplary suture line configuration shown in FIG. 11A taken along line 11B-11B.

FIG. 12 is a perspective view of another example of a prosthetic heart valve comprising a frame and an outer skirt releasably attached to an inflow end of the frame, in accordance with the present disclosure.

FIG. 13 is a perspective view of another example of a prosthetic heart valve comprising a frame, a first outer skirt releasably attached at an inflow end of the frame, and a second outer skirt releasably attached at the outflow end of the frame, in accordance with the present disclosure.

FIG. 14 is an illustration of the exemplary heart valve of FIG. 13 implanted within a native heart valve.

FIG. 15 is a perspective view of another example of a prosthetic heart valve comprising a frame, a first outer skirt attached at an inflow end of the frame via bioresorbable couplers, and a second outer skirt attached at the outflow end of the frame via bioresorbable couplers, in accordance with the present disclosure.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

Prosthetic valves disclosed herein can be radially compressible and expandable between a radially compressed state and a radially expanded state. Thus, the prosthetic valves can be crimped on or retained by an implant delivery apparatus in the radially compressed state during delivery, and then expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail late

### Examples of the Disclosed Technology

As discussed above, for treatment of AI patients, it may be desirable or necessary to utilize an oversized prosthetic valve without an outer skirt, while it may be desirable or necessary to utilize a relatively smaller prosthetic valve having an outer skirt configured to form a seal between the calcified native valve and the prosthetic valve for treatment of AS patients. Thus, in order to properly treat both AI and AS patients with conventional prosthetic heart valves, a hospital might be required to carry multiple types of prosthetic valves, each in various sizes.

Further, after a period of use of an implanted prosthetic heart valve, it may be desirable or necessary to explant the prosthetic valve so that a new prosthetic valve can be implanted. Such explant procedures can be complicated for a surgeon to perform and/or traumatic to the patient depending on the anatomy of the patient. For example, overtime soft portions of the prosthetic heart valve, such as an outer skirt, may experience tissue ingrowth. During explant, the ingrown tissue may make it difficult for the prosthetic valve to be removed from the implantation site, which may thereby make the procedure more complex for the surgeon to perform, may increase the time required to perform the surgical procedure, and/or may require significant cutting or damage to the surrounding tissue (or other tissues) as the surgeon attempts to remove the prosthetic valve structure from the patient.

Accordingly, a prosthetic heart valve that can facilitate explant procedures is desired. For example, there is a need for prosthetic heart valves and explant procedures that limit tissue trauma and improve the ease and/or expedite of the removal process.

Also, a prosthetic heart valve that can be implanted in a larger patient population, including patients suffering from AS and AI, is desired. For example, there is a need for prosthetic heart valves that can be used in either of AS or AI patients.

Described herein are examples of prosthetic heart valves including an annular frame, a plurality of leaflets (that is, a valvular structure) attached to the frame, an outer skirt, and optionally an inner skirt. The outer skirt can be attached or secured to the frame in a detachable manner that allows the frame to be easily separated or released from the skirt, for example, prior to an implant procedure of a prosthetic heart valve or during explant of a prosthetic heart valve. In some examples, the leaflets can be attached to the frame via commissures formed by joining pairs of adjacent ends (for example, commissure tabs) of the leaflets. The cusp edges of the leaflets can be attached to the outer skirt, an inner skirt mounted to an inner surface of the frame, or directly to selected struts of the frame. In some examples, the frame can be radially expandable and compressible, and can be configured for transcatheter implantation.

In some examples, the outer skirt can be attached to any of the frame, an inner skirt, and/or the leaflet structure via a plurality of looped retaining sutures, and the looped retaining sutures are formed around struts of the frame (or another point of attachment on the prosthetic valve) and over a pull suture that extends along a suture line. One or more ends of the pull suture can extend beyond an edge of the skirt and/or frame and can be temporarily attached to the frame (or another point of attachment on the prosthetic valve).

Prior to an implant procedure or during an explant procedure, a surgeon can free an end of the pull suture from the frame and, by pulling on the loose end, withdraw the pull suture from the suture line to release the frame from the outer skirt, leaving the retaining sutures attached to the frame. In some examples, the outer skirt has a scalloped form corresponding to a number of leaflets in the valvular structure. The outer skirt can be a single piece of material (for example, a voluminous material or a relatively flat fabric layer) that extends completely around the frame or can include multiple portions, and each scalloped portion can include a separate pull suture for releasing the corresponding portion of the outer skirt from the frame. In other examples, a single pull suture can be configured to release the outer skirt from the frame.

In this way, a prosthetic valve can be used to treat a variety of heart conditions, such as for treating diseased valves in both AI and AS patients. In some examples, a prosthetic heart valve can include an outer skirt that is selectively removable prior to implantation of the prosthetic valve depending on the anatomy and condition of the patient. In some examples, the outer skirt may be specifically configured to form a seal between an irregularly shaped or calcified native annulus in AS patients, which can be removed from the prosthetic valve to convert the prosthetic valve for use in AI patients, in which the outer skirt may not be needed. In certain examples, the outer skirt an include one or more fabric portions configured to cover an outer circumference of a frame of the prosthetic valve and enable tissue ingrowth therein. Accordingly, a hospital or health care provider can carry one type of heart valve in a range of sizes for treating both AI patients and AS patients.

Further, during an exemplary explant procedure, a surgeon can easily separate the frame from the outer skirt which may have tissue ingrown therein, thereby resulting in release and removal of the frame from the outer skirt and the implantation site. In some examples, the removal of the frame can provide better access to the implantation site for cutting and removal of the outer skirt and/or the native leaflets, thereby making the explant process less traumatic to the surrounding tissue. In some examples, in transcatheter explant procedures, the released frame can be radially compressed to a smaller diameter relative to a diameter of the prosthetic valve including the outer skirt, which can improve ease of transcatheter removal.

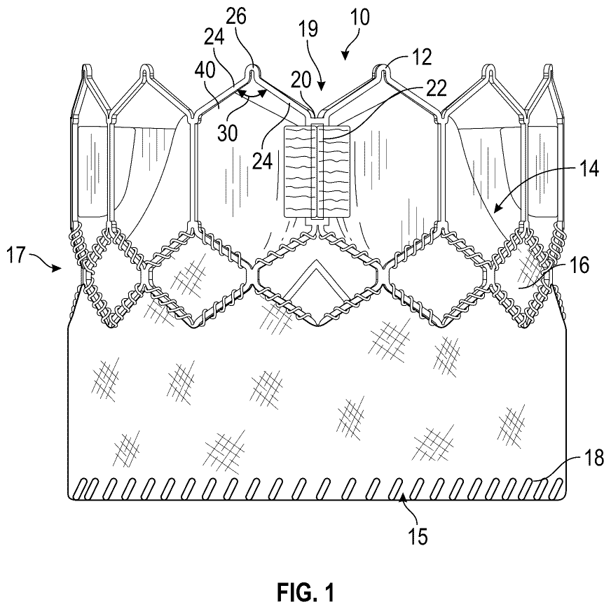

FIG. 1 shows one example of a prosthetic heart valve 10. Any of the prosthetic valves disclosed herein are adapted to be implanted in the native aortic annulus, although in other examples they can be adapted to be implanted in the other native annuluses of the heart (for example, the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve).

The prosthetic valve 10 can have four main components: a stent or frame 12, a valvular structure 14, an inner skirt 16, and a perivalvular outer sealing member or outer skirt 18. The prosthetic valve 10 can have an inflow end portion 15, an intermediate portion 17, and an outflow end portion 19. The inner skirt 16 can be arranged on and/or coupled to an inner surface of the frame 12 while the outer skirt 18 can be arranged on and/or coupled to an outer surface of the frame 12.

The valvular structure 14 can comprise three leaflets 40, collectively forming a leaflet structure, which can be arranged to collapse in a tricuspid arrangement, although in other examples there can be greater or fewer number of leaflets (for example, one or more leaflets 40). The leaflets 40 can be secured to one another at their adjacent sides to form commissures 22 of the valvular structure 14. The lower edge of valvular structure 14 can have an undulating, curved scalloped shape and can be secured to the inner skirt 16 by sutures (not shown). In some examples, the leaflets 40 can be formed of pericardial tissue (for example, bovine pericardial tissue), biocompatible synthetic materials, or various other suitable natural or synthetic materials as known in the art and described in U.S. Pat. No. 6,730,118, which is incorporated by reference herein.

The frame 12 can be radially compressible (collapsible) and expandable (for example, expanded configuration shown in FIG. 1) and comprise a plurality of interconnected struts 24. A plurality of apices 26 that are spaced circumferentially apart are formed at the inflow end portion 15 and the outflow end portion 19 of the frame 12 (only the apices 26 at the outflow end portion 19 are visible in FIG. 1). Each apex 26 is formed at a junction between two angled struts 24 at either the inflow end portion 15 or the outflow end portion 19. FIG. 1 depicts a known frame design with apices 26 that form a U-shaped bend between the two angled struts 24. In some examples, an angle 30 between the two angled struts 24, connected at the apex 26, can be in a range of 90 to 120 degrees.

The frame 12 can be formed with a plurality of circumferentially spaced slots, or commissure windows 20 that are adapted to mount the commissures 22 of the valvular structure 14 to the frame. The frame 12 can be made of any of various suitable plastically-expandable materials (for example, stainless steel, etc.) or self-expanding materials (for example, nickel titanium alloy (NiTi), such as nitinol), as known in the art. When constructed of a plastically-expandable material, the frame 12 (and thus the prosthetic valve 10) can be crimped to a radially collapsed configuration on a delivery catheter or apparatus and then expanded inside a patient by an inflatable balloon or equivalent expansion mechanism. When constructed of a self-expandable material, the frame 12 (and thus the prosthetic valve 10) can be crimped to a radially collapsed configuration and restrained in the collapsed configuration by insertion into a sheath or equivalent mechanism of a delivery catheter. Once inside the body, the prosthetic valve can be advanced from the delivery sheath, which allows the prosthetic valve to expand to its functional size.

Suitable plastically-expandable materials that can be used to form the frames disclosed herein (such as, for examples the frame 12) include, metal alloys, polymers, or combinations thereof. Example metal alloys can comprise one or more of the following: nickel, cobalt, chromium, molybdenum, titanium, or other biocompatible metal. In some examples, the frame 12 can comprise stainless steel. In some examples, the frame 12 can comprise cobalt-chromium. In some examples, the frame 12 can comprise nickel-cobalt-chromium. In some examples, the frame 12 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

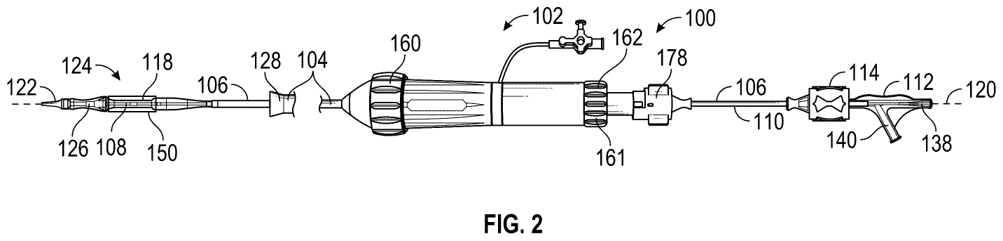

FIG. 2 shows an example of a delivery apparatus 100 that can be used to implant an expandable prosthetic heart valve (for example, prosthetic heart valve 10 of FIG. 1 or any of the other prosthetic heart valves described herein). In some examples, the delivery apparatus 100 is specifically adapted for use in introducing a prosthetic valve into a heart.

The delivery apparatus 100 in the illustrated example of FIG. 2 is a balloon catheter comprising a handle 102 and a steerable, outer shaft 104 extending distally from the handle 102. The delivery apparatus 100 can further comprise an intermediate shaft 106 (which also may be referred to as a balloon shaft) that extends proximally from the handle 102 and distally from the handle 102, the portion extending distally from the handle 102 also extending coaxially through the outer shaft 104. Additionally, the delivery apparatus 100 can further comprise an inner shaft 108 extending distally from the handle 102 coaxially through the intermediate shaft 106 and the outer shaft 104 and proximally from the handle 102 coaxially through the intermediate shaft 106.

The outer shaft 104 and the intermediate shaft 106 can be configured to translate (for example, move) longitudinally, along a central longitudinal axis 120 of the delivery apparatus 100, relative to one another to facilitate delivery and positioning of a prosthetic valve at an implantation site in a patient's body.

The intermediate shaft 106 can include a proximal end portion 110 that extends proximally from a proximal end of the handle 102, to an adaptor 112. A rotatable knob 114 can be mounted on the proximal end portion 110 and can be configured to rotate the intermediate shaft 106 around the central longitudinal axis 120 and relative to the outer shaft 104.

The adaptor 112 can include a first port 138 configured to receive a guidewire therethrough and a second port 140 configured to receive fluid (for example, inflation fluid) from a fluid source. The second port 140 can be fluidly coupled to an inner lumen of the intermediate shaft 106.

The intermediate shaft 106 can further include a distal end portion that extends distally beyond a distal end of the outer shaft 104 when a distal end of the outer shaft 104 is positioned away from an inflatable balloon 118 of the delivery apparatus 100. A distal end portion of the inner shaft 108 can extend distally beyond the distal end portion of the intermediate shaft 106.

The balloon 118 can be coupled to the distal end portion of the intermediate shaft 106.

In some examples, a distal end of the balloon 118 can be coupled to a distal end of the delivery apparatus 100, such as to a nose cone 122 (as shown in FIGS. 2), or to an alternate component at the distal end of the delivery apparatus 100 (for example, a distal shoulder). An intermediate portion of the balloon 118 can overlay a valve mounting portion 124 of a distal end portion of the delivery apparatus 100 and a distal end portion of the balloon 118 can overly a distal shoulder 126 of the delivery apparatus 100. The valve mounting portion 124 and the intermediate portion of the balloon 118 can be configured to receive a prosthetic heart valve in a radially compressed state. For example, as shown schematically in FIG. 2, a prosthetic heart valve 150 (which can be one of the prosthetic valves described herein) can be mounted around the balloon 118, at the valve mounting portion 124 of the delivery apparatus 100.

The balloon shoulder assembly, including the distal shoulder 126, is configured to maintain the prosthetic heart valve 150 (or other medical device) at a fixed position on the balloon 118 during delivery through the patient's vasculature.

The outer shaft 104 can include a distal tip portion 128 mounted on its distal end. The outer shaft 104 and the intermediate shaft 106 can be translated axially relative to one another to position the distal tip portion 128 adjacent to a proximal end of the valve mounting portion 124, when the prosthetic valve 150 is mounted in the radially compressed state on the valve mounting portion 124 (as shown in FIG. 2) and during delivery of the prosthetic valve to the target implantation site. As such, the distal tip portion 128 can be configured to resist movement of the prosthetic valve 150 relative to the balloon 118 proximally, in the axial direction, relative to the balloon 118, when the distal tip portion 128 is arranged adjacent to a proximal side of the valve mounting portion 124.

An annular space can be defined between an outer surface of the inner shaft 108 and an inner surface of the intermediate shaft 106 and can be configured to receive fluid from a fluid source via the second port 140 of the adaptor 112. The annular space can be fluidly coupled to a fluid passageway formed between the outer surface of the distal end portion of the inner shaft 108 and an inner surface of the balloon 118. As such, fluid from the fluid source can flow to the fluid passageway from the annular space to inflate the balloon 118 and radially expand and deploy the prosthetic valve 150.

An inner lumen of the inner shaft can be configured to receive a guidewire therethrough, for navigating the distal end portion of the delivery apparatus 100 to the target implantation site.

The handle 102 can include a steering mechanism configured to adjust the curvature of the distal end portion of the delivery apparatus 100. In the illustrated example, the handle 102 includes an adjustment member, such as the illustrated rotatable knob 160, which in turn is operatively coupled to the proximal end portion of a pull wire. The pull wire can extend distally from the handle 102 through the outer shaft 104 and has a distal end portion affixed to the outer shaft 104 at or near the distal end of the outer shaft 104. Rotating the knob 160 can increase or decrease the tension in the pull wire, thereby adjusting the curvature of the distal end portion of the delivery apparatus 100. Further details on steering or flex mechanisms for the delivery apparatus can be found in U.S. Pat. No. 9,339,384, which is incorporated by reference herein.

The handle 102 can further include an adjustment mechanism 161 including an adjustment member, such as the illustrated rotatable knob 162, and an associated locking mechanism including another adjustment member, configured as a rotatable knob 178. The adjustment mechanism 161 is configured to adjust the axial position of the intermediate shaft 106 relative to the outer shaft 104 (for example, for fine positioning at the implantation site). Further details on the delivery apparatus 100 can be found in U.S. Provisional Application Nos. 63/069,567 and 63/138,890, which are each incorporated by reference herein.

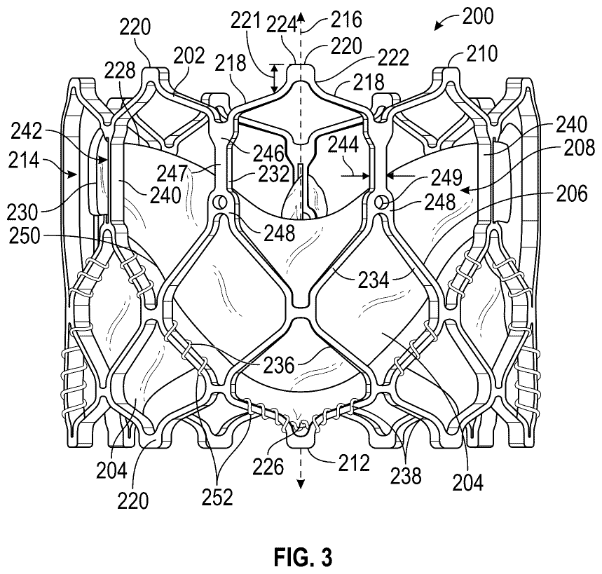

FIG. 3 shows an example of a prosthetic heart valve 200 comprising a radially expandable and compressible annular frame 202 and a plurality of leaflets 204 secured to the frame. Each leaflet 204 can comprise opposing commissure tabs disposed on opposite sides of the leaflet 204 and a cusp edge portion extending between the opposing commissure tabs. Further, the leaflets can include an inflow end 226 and an outflow end 228.

The frame 202 can be made of any of various suitable plastically-expandable materials (for example, stainless steel, etc.) or self-expanding materials (for example, nickel titanium alloy (NiTi), such as nitinol), as known in the art. In some examples, the frame 202 comprises a plastically-expandable material, such as those described above with reference to the prosthetic heart valve 10 of FIG. 1.

The frame 202 can comprise a plurality of interconnected struts 206 which form multiple rows of open cells 208 between an outflow end 210 and an inflow end 212 of the frame 202. In some examples, as shown in FIG. 3, the frame 202 can comprise three rows of cells 208 with a first (for example, upper in FIG. 3) row of cells 214, disposed at the outflow end 210, having cells 208 that are elongated in an axial direction (relative to a central longitudinal axis 216 of the frame 202), as compared to cells 208 in the remaining rows of cells. For example, the cells 208 of the first row of cells 214 can have a longer axial length, defined in a direction of a central longitudinal axis 216 of the frame 202, than cells 208 in the remaining rows of cells (for example, cells in the row of cells at the inflow end 212).

In some examples, as shown in FIG. 3, each row of cells 208 comprises nine cells. Thus, in such examples, the frame 202 can be referred to as a nine-cell frame.

In other examples, the frame 202 can comprise more than three rows of cells (for example, four or five) and/or more or less than nine cells per row. In some examples, the cells 208 in the first row of cells 214 may not be elongated compared to cells 208 in remaining rows of cells of the frame 202.

The interconnected struts 206 can include a plurality of angled struts 218, 234, 236, and 238 arranged in a plurality of rows of circumferentially extending rows of angled struts, with the rows being arrayed along the length of the frame between the outflow end 210 and the inflow end 212 of the frame 202. For example, the frame 202 can comprise a first row of angled struts 238 arranged end-to-end and extending circumferentially at the inflow end 212 of the frame; a second row of circumferentially extending, angled struts 236; a third row of circumferentially extending, angled struts 234; and a fourth row of circumferentially extending, angled struts 218 at the outflow end 210 of the frame 12. The fourth row of angled struts 218 can be connected to the third row of angled struts 234 by a plurality of axially extending window strut portions 240 and a plurality of axial (for example, axially extending) struts 232. The axially extending window strut portions 240 define commissure windows (for example, open windows) 242 that are spaced apart from one another around the frame 202, in a circumferential arrangement, and which are adapted to receive a pair of commissure tabs of a pair of adjacent leaflets 204 formed into a commissure 230.

One or more (for example, two, as shown in FIG. 3) axial struts 232 can be positioned between, in the circumferential direction, two commissure windows 242 formed by the window strut portions 240. Since the frame 202 can include fewer cells per row (for example, nine) and fewer axial struts 232 between each commissure window 242, as compared to other prosthetic heart valves, such as the prosthetic heart valve 10 of FIG. 1, each cell 208 can have an increased width (in the circumferential direction), thereby providing a larger opening for blood flow and/or coronary access.

Each axial strut 232 and each window strut portion 240 extends from a location defined by the convergence of the lower ends (for example, ends arranged inward of and farthest away from the outflow end 210) of two angled struts 218 (which can also be referred to as an upper strut junction or upper elongated strut junction) to another location defined by the convergence of the upper ends (for example, ends arranged closer to the outflow end 210) of two angled struts 234 (which can also be referred to as a lower strut junction or lower elongate strut junction). Each axial strut 232 and each window strut portion 240 forms an axial side of two adjacent cells of the first row of cells 214.

In some examples, as shown in FIG. 3, each axial strut 232 can have a width 244 that is larger than a width of the angled struts 218, 234, 236, and/or 238. As used herein, a “width” of a strut is measured between opposing locations on opposing surfaces of a strut that extend between the radially facing inner and outer surfaces of the strut (relative to the central longitudinal axis 216 of the frame 202). A “thickness” of a strut is measured between opposing locations on the radially facing inner and outer surfaces of a strut and is perpendicular to the width of the strut.

Commissure tabs of adjacent leaflets 204 can be secured together to form commissures 230. Each commissure 230 of the prosthetic heart valve 200 comprises two commissure tabs paired together, one from each of two adjacent leaflets 204, and extending through a commissure window 242 of the frame 202. Each commissure 230 can be secured to the window strut portions 240 forming the commissure window 242. Further details regarding the attachment of the commissures 230 to the windows 242 are described in U.S. Pat. No. 9,393,110, previously incorporated herein.

The cusp edge portion (for example, scallop edge) of each leaflet 204 can be secured to the frame via one or more fasteners (for example, sutures). In some examples, as shown in FIG. 3, the cusp edge portion of each leaflet 204 can be secured directly to the struts of the frame 202 (for example, angled struts 234, 236, and 238). For example, the cusp edge portions 250 of the leaflets 204 can be connected to the angled struts 234, 236, 238 that generally follow the contour of the cusp edge portions of the leaflets via sutures 252.

In some examples, the cusp edge portions 250 of the leaflets 204 can be secured to an inner skirt and the inner skirt can then be secured directly to the frame 202.

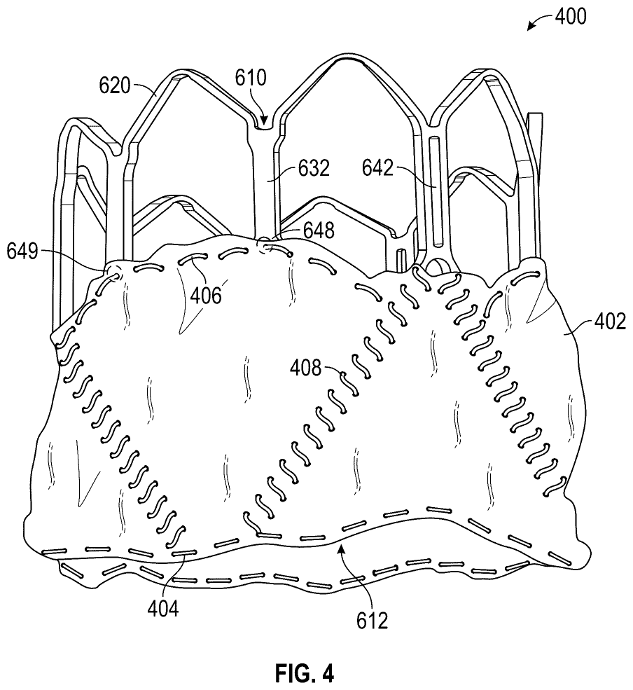

Further, in some examples, an outer skirt can be connected to an outer surface of the frame 202 (for example, similar to the outer skirt 18 of the valve 10 of FIG. 1 or the outer skirt 402 of the valve 400 shown in FIG. 4 and discussed below).

As illustrated in FIG. 3, in some examples, one or more of or each of the axial struts 232 can comprise an inflow end portion (for example, inflow end portion that is closer to the inflow end than the outflow end portion 246) 248 that is widened relative to a middle portion 247 of the axial strut 232 (which can be defined by the width 244), similar to the outflow end portion 246 (as described above). In some examples, the inflow end portion 248 of the axial strut 232 can comprise an aperture 249. The apertures 249 can be configured to receive fasteners (for example, sutures) for attaching soft components of the prosthetic heart valve 200 to the frame 202. For example, in some examples, an outer skirt can be positioned around an outer surface of the frame 202 and secured to the apertures 249 and/or other components of the frame (for example, as shown valve 400 in FIG. 4 and described below).

The frame 202 can further comprise a plurality of apices 220 formed at the inflow end 212 and the outflow end 210, each apex 220 forming a junction between two angled struts 218 at the inflow end 212 or outflow end 210. As such, the apices 220 are spaced apart from one another, in a circumferential direction at the inflow end 212 and the outflow end 210. As shown in FIG. 3, each apex 220 can have side portions 222 that curve or bend axially outward from the angled strut 218 to which it is connected and an end portion 224 that extends between the two side portions 222 of the apex 220. The side portions 222 can extend in a direction that is parallel to the central longitudinal axis 216. The end portion 224 can be relatively flat and include a surface that is disposed normal to the central longitudinal axis 216. Each apex 220 can have two bends at its end portion 224 and two bends at the side portions 222 (for example, one at the junction between each side portion 222 and angled strut 218). In this way, the apices 220 can be U-shaped, similar to the apices 26 of the valve of FIG. 1.

FIG. 4 is a perspective view of an example of a prosthetic heart valve 400 comprising a frame 602 and an outer (fabric or other biocompatible material) skirt 402 secured to the frame 602. It will be appreciated that the frame 602 is merely exemplary, and can be similar to or include features of any one of the frames described herein. For example, the frame 602 can have features similar to (or can be replaced by) any of the frames described herein, such as frame 202 illustrated in FIG. 3. As shown in FIG. 4, the outer skirt 402 can extend around an outer surface of the frame 602, from the inflow end 612 toward the outflow end 610. In some examples, as shown in FIG. 4, the outer skirt 402 can be secured to struts of the frame at the inflow end 612 by one or more sutures 404. The leaflets of the prosthetic valve are omitted for purposes of illustration, but can be the same as leaflets 204 of the prosthetic valve 200.

Further, in some examples, an outflow end of the outer skirt 402 can be secured to inflow end portions 648 of the axial struts 632 and/or to upper or outflow ends of the angled struts (not shown) by one or more sutures 406. In some examples, a portion of the sutures 404 can extend through and be secured to the apertures 649 in the inflow end portions 648 of the axial struts 632 (apertures 649 shown in dashed lines in FIG. 4 to denote their position underneath the outer skirt 402). In some examples, one or more of the sutures 404 and/or 406 can be in-and-out stitches or other types of stitches (for example, chain stitches, cross stitches, etc.).

In some examples, as shown in FIG. 4, additional sutures 408, which can be configured as whip stitches, can secure the outer skirt 402 to angled struts (not shown) of the frame 602 disposed and extending between the inflow end 612 and inflow ends of window strut portions forming the commissure windows 642 of the frame 602.

Additional features and related examples of the foregoing prosthetic valves (as well as other prosthetic valves and techniques for implantation associated therewith) that can be used in combination with the exemplary detachable outer skirts disclosed herein are described in U.S. Provisional Patent Application No. 63/178,416, which is incorporated by reference herein.

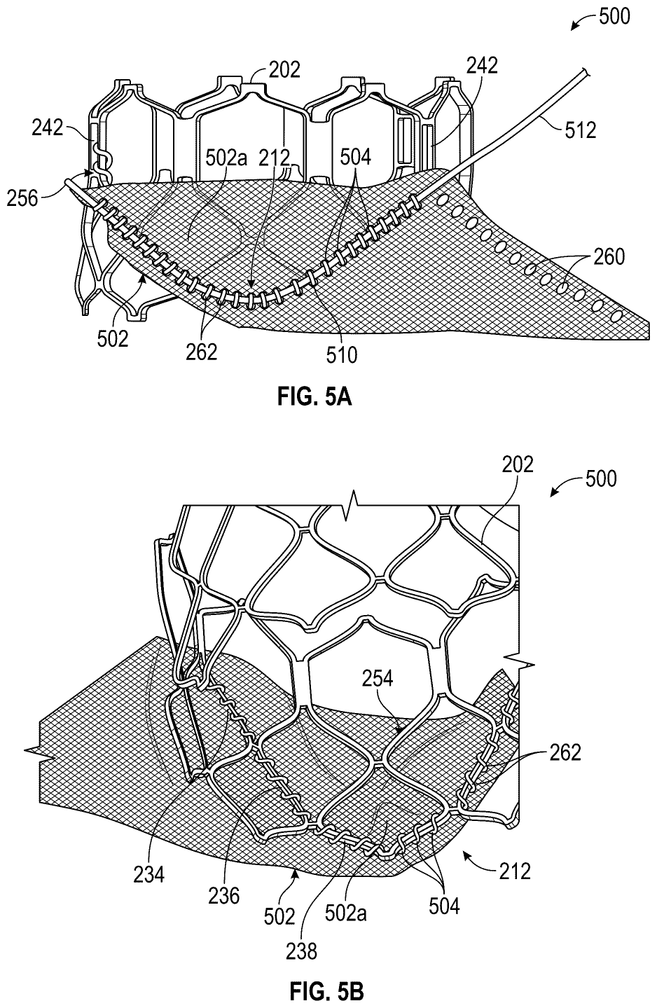

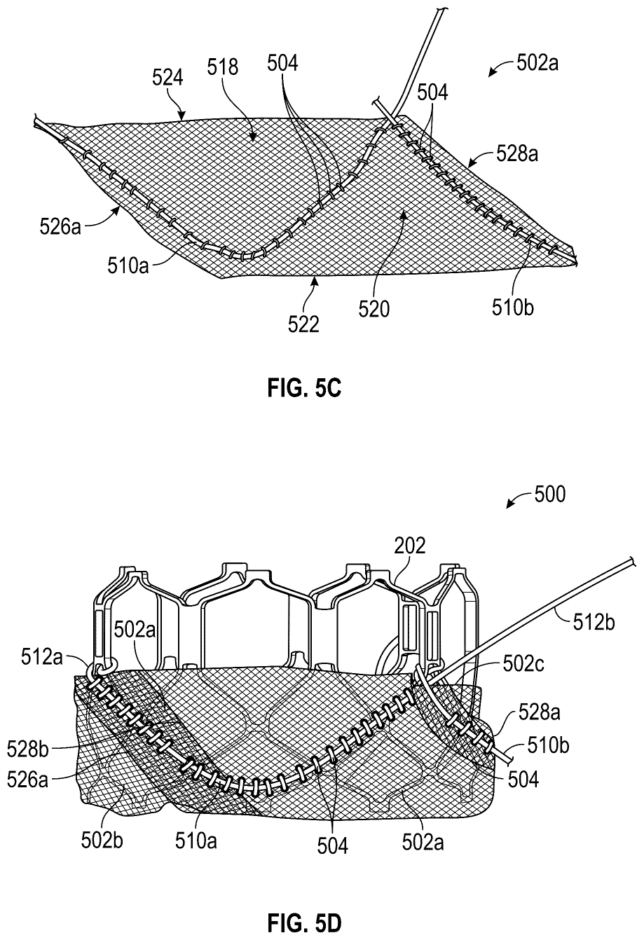

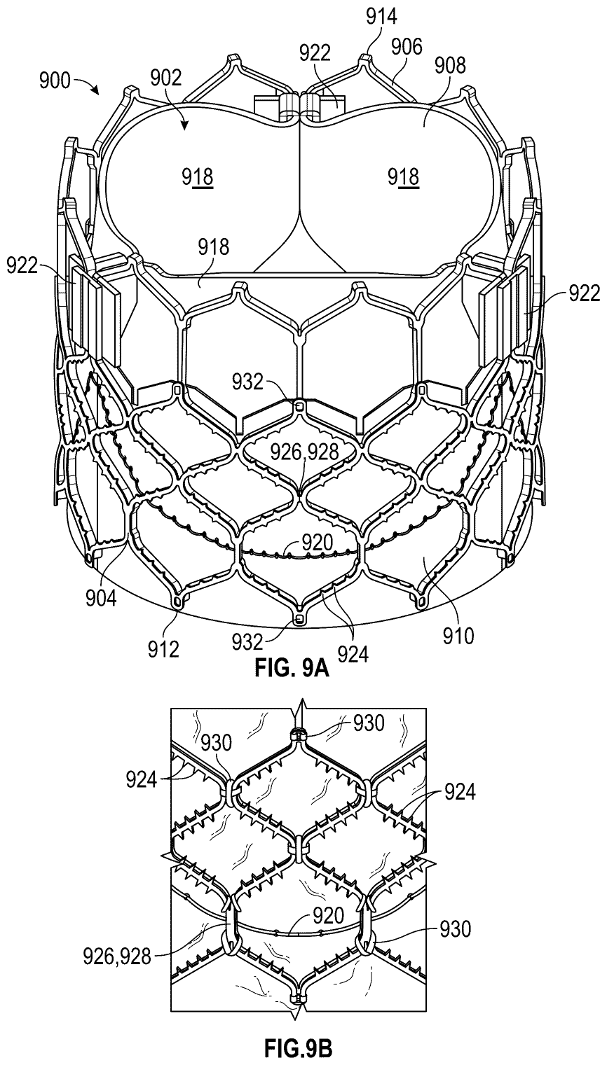

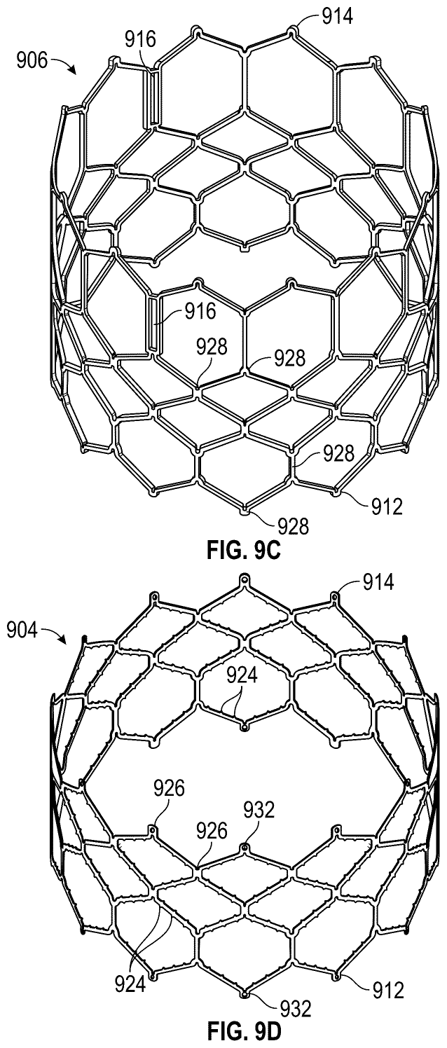

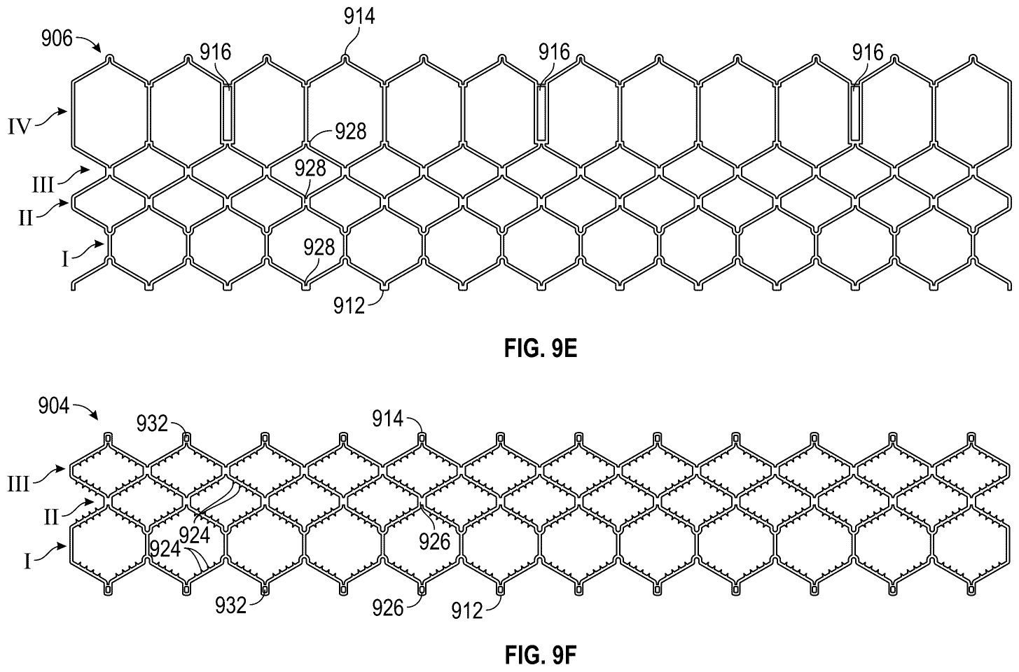

Turning now to FIGS. 5A and 5B, an example of a prosthetic heart valve 500 comprising the frame 202 and an outer (fabric or other biocompatible material) skirt 502 secured to the frame 202 is shown and described. It will be appreciated that the frame 202 can have features similar to or be replaced by another frame, such as for example, the frame 12 shown in FIG. 1, the frame 602 shown in FIG. 4, frames 904, 906 shown in FIGS. 9A-9F (described below), and/or other frames, such as those described in U.S. Provisional Patent Application No. 63/178,416, previously incorporated herein, and International Application No. PCT/US2021/034399, which is incorporated herein by reference. Further, the skirt 502 can include features similar to or be replaced by another outer skirt, such as for example, the outer skirt 18 shown in FIG. 1, the outer skirt 402 shown in FIG. 4, and/or other outer skirts, such as those described in U.S. Pat. Nos. 9,393,110 and 11,096,781, each of which is incorporated by reference herein. The soft components on the interior of the prosthetic valve 500, such as the leaflets 204 and an optional inner skirt, are not shown in FIGS. 5A-5B for purposes of illustration.

Different from conventional prosthetic heart valves, the prosthetic heart valve 500 is configured such that the outer skirt 502 can be readily detachable, so that, for example, the outer skirt can be removed prior to implantation of the prosthetic valve in an AI patient. In another example, during explant of a prosthetic valve that was implanted with the outer skirt attached thereto, the frame can be easily removed from the outer skirt to facilitate removal of the prosthetic valve from the implantation site.

Specifically, as can be seen in FIGS. 5A and 5B, the outer skirt 502 can be releasably attached to the frame via a retaining and pull suture mechanism comprising one or more retaining sutures 504 sutured to or looped around selected struts of the frame 202, such as struts 234, 236, and 238, which are disposed between the inflow end 212 of the frame and inflow ends of window strut portions forming the commissure windows 242 of the frame 202. In some examples, the retaining sutures 504 are formed as looped or whip stitches 262 that extend through holes or apertures 260 in the material of the outer skirt 502, over a pull suture 510, and around an interior surface 254 of the angled struts, thereby sandwiching or capturing the material of the outer skirt 502 between the pull suture 510 and an exterior surface 256 of the angled struts. In some examples, the stitches 262 can also extend through an inner skirt (not shown) and/or the cusp edge portions 250 of the leaflets 204 to secure those components to the frame.

In some examples, one or both free ends of the pull suture 510, such as a free end 512 shown in FIG. 5A, can extend beyond an edge of the outer skirt 502 and/or the frame 202, and can be attached to the frame and/or the outer skirt, such as attachment via an adhesive, a stitch, tying or knotting the free end 512 to an adjacent strut, an elastic band, and/or other releasable retaining mechanism. In certain examples, both ends of the pull suture 510 are secured to the frame. During an explant procedure, the secured end(s) 512 of the pull suture 510 can be freed from its attachment to the frame, after which it can be pulled through the retaining sutures 504 along the suture line to free the frame 202 from the outer skirt 502 (or a portion thereof), as further described below.

In some examples, the outer skirt 502 can include multiple separate portions or sections. For example, the outer skirt can include multiple similarly shaped and configured sections corresponding to in number to the number of leaflets in a valvular structure attached to the frame (such as, for example, leaflets 204 illustrated in FIG. 3). As discussed above and shown in FIG. 3, the cusp edge portion 250 of each leaflet 204 can be secured directly to the struts of the frame 202 (for example, angled struts 234, 236, and 238). For example, the cusp edge portions 250 of the leaflets can be sutured to selected ones of the angled struts 234, 236, and 238 that generally follow the contour of the cusp edges of the leaflets. Accordingly, in certain examples, the separate portions of the outer skirt 502 can be sutured along a similar suture line or to the same angled struts 234, 236, and 238 as the corresponding leaflet. In one specific example, the valvular structure can include three leaflets and the outer skirt can include three skirt portions similar in shape and size that are sutured to the exterior of the frame to align with the positions of the leaflets on the frame interior. It will be appreciated, however, that in alternate examples the outer skirt portions can be offset relative to the positions of leaflet attachment, the outer skirt can include more of fewer skirt portions relative to the number of leaflets in a valvular structure, and/or the outer skirt can comprise a single piece of material.

An example of a skirt portion 502a of the outer skirt 502 (which includes a total of, for example, three similarly configured skirt portions) is shown in FIG. 5C. For illustrative purposes, the skirt portion 502a is shown unattached from a frame and in a flat configuration (rather than being wrapped over an exterior surface of an annular frame). Also for illustrative purposes, the skirt portion 502a is shown with the retaining sutures 504 and pull sutures 510a and 510b sutured thereto, although in practice the foregoing sutures can be attached or stitched into the outer skirt while it is disposed on the exterior surface of the frame in order to extend or loop the retaining sutures around the angled struts of the frame.

As can be seen in FIG. 5C, the skirt portion 502a can have a generally trapezoidal shape including a first covering section 518 and a second covering section 520. In the present example, the first section 518 of the skirt portion 502a is disposed between descending and ascending portions of the pull suture 510a, and therefore can be configured to cover a section of the frame corresponding to a region of attachment of a cusp edge of one of the leaflets of the valvular structure on the interior of a frame. For example, in an example where a prosthetic heart valve includes the frame 202, the section 518 of the skirt portion 502a can be configured to cover selected ones of the angled struts 234, 236, and 238 that are aligned with the cusp edge of one of the leaflets (similar to, for example, the cusp edge 250 of the leaflets 204 shown in FIG. 3). Also, when mounted on the frame 202, the section 518 of the skirt portion 502a can face an outer surface of a corresponding leaflet.

Also in the example of FIG. 5C, the second section 520 of the skirt portion 502a is disposed between the ascending portion of pull suture 510a and a descending portion of the pull suture 510b, and therefore can be configured to cover a section of the frame corresponding to an area that is between the cusp edges of adjacent ones of the leaflets. For example, when used with the frame 202, the second section 520 of the skirt portion 502a can be configured to cover selected ones of the angled struts 234, 236, and 238 that are between adjacent ones of the leaflets.

In certain examples, an inflow edge 522 of the skirt portion 502a can be oriented toward or disposed on or proximate to an inflow end of the frame, while an outflow edge 524 thereof is oriented toward the outflow end of the frame. A first side edge 526a can be configured to overlap with a side edge of an adjacent (second) skirt portion and have a section of the suture line corresponding the descending portion of the pull suture 510a sutured therethrough. Thus, the first side edge 526a is configured to be detached from the frame via removal of the pull suture 510a from the suture line. A second side edge 528a, which opposes the first side edge 526a, can be configured to overlap with a side edge of an adjacent (third) skirt portion and have a section of the suture line corresponding to the descending portion of the pull suture 510b sutured therethrough. Thus, the second side edge 528a is configured to be detached from the frame via removal of the pull suture 510b from the suture line.

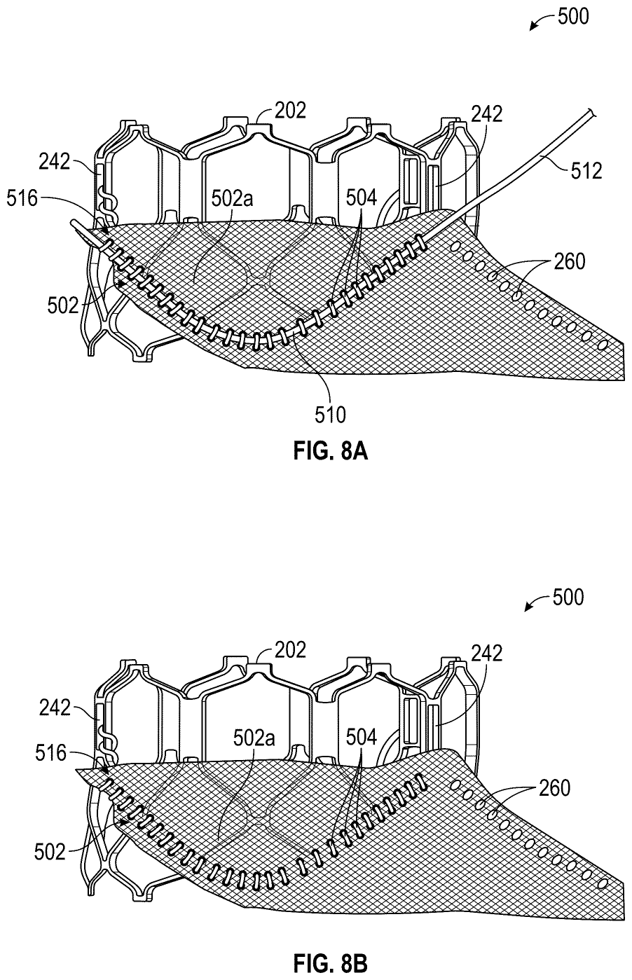

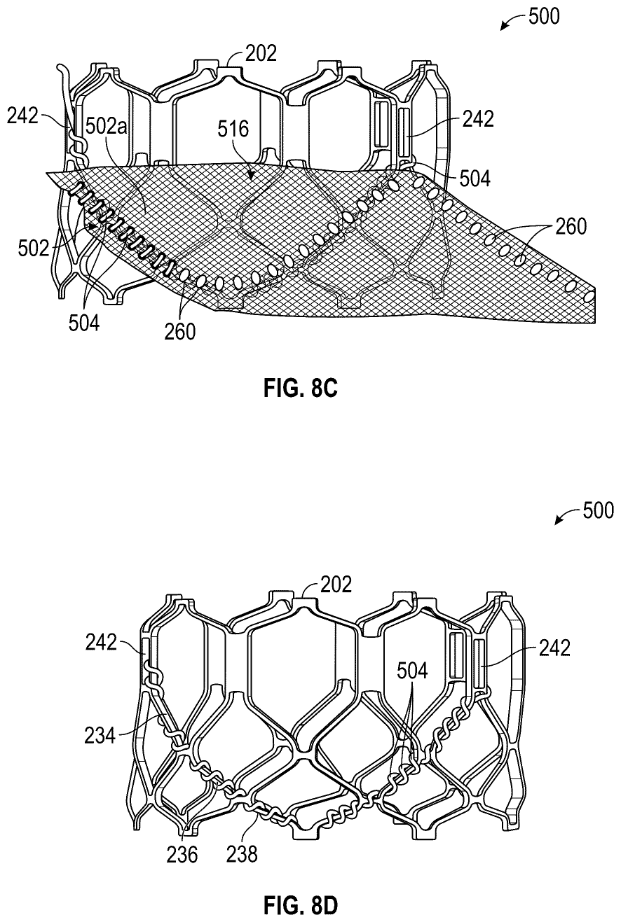

For simplicity, a single portion of the outer skirt 502, the skirt portion 502a, is illustrated in FIGS. 5A-5C and FIGS. 8A-8C. It will be appreciated that additional portions (for example, two additional portions) can be similarly releasably attached to the frame such that the outer skirt substantially encompasses at least the inflow end portion of the frame. Accordingly, in such examples, when all of the skirt portions of the outer skirt are mounted to a frame, the skirt portions can have overlapping side edges and the overlapping edges can be detached from the frame via removal of the respective pull sutures threaded through the retaining sutures along the suture line. As edges of the skirt portion can be overlapping in the foregoing examples, each pull suture can be configured to release a first side edge of a first skirt portion and a second side edge of a second skirt portion.

For example, FIG. 5D illustrates an example where three skirt portions 502a, 502b, and 502c of the outer skirt 502 are mounted to the frame 202. As can be seen therein, a first side edge 526a of the skirt portion 502a can overlap with a second side edge 528b of the second skirt portion 502b. In a fully assembled prosthetic valve, each skirt portion 502 has a respective pull suture 510 and at least one retaining suture 504 forming a plurality of stitches 262. The opposing ends 512a, 512b of each pull suture 510 can be secured to the frame at convenient locations. For example, the end 512a can be knotted or tied off to a strut adjacent a commissure window, as shown. The other end 512b is shown detached from the frame for purposes of illustration, but can be similarly secured to the frame in the fully assembled configuration of the prosthetic valve. When the ends 512a, 512b are detached from the frame in an explant procedure, the pull suture 510 can be pulled through the retaining sutures 504 and away from the respective skirt portion 502. Accordingly, removal or withdraw of the pull suture 510a from the stitches 262 along the suture line can enable detachment of the skirt portion 502a from the frame. In order for the skirt portion 502c to be released from the frame, the second pull suture 510b can be withdrawn from the suture line. It will be appreciated that, although not specifically shown, each the skirt portions can be similarly configured to the outer skirt portion 502a shown in FIG. 5C.

As discussed above, in additional or alternate examples, the outer skirt can include a single piece of material, include more of fewer portions, and/or the outer skirt portions can have a different positioning or orientation on the frame. Further, in additional or alternate examples, the outer skirt portions can have other shapes, such as alternating U-shaped or V-shaped portions or other shapes that correspond to a shape of a frame and/or a shape of leaflets or an inner skirt of a valvular structure. Furthermore, in additional or alternate examples, the outer skirt portions can be non-overlapping or overlap to a greater degree than the illustrated example. Further still, in additional or alternate examples, the outer skirt can be releasably attached by more or fewer pull sutures (for example, each portion of the outer skirt can be attached by multiple pull sutures for finer control of frame release during explant of a valve, or all portions of the outer skirt can be attached to the frame with a single pull suture such that the surgeon need only locate and pull a single end of a pull suture to release the frame from the outer skirt) and/or the pull sutures can have a different configuration and/or orientation (for example, a single pull suture enabling detachment of a single portion of the outer skirt, or one or more ends can be oriented toward the inflow end). In a specific example, the outer skirt can comprise a single piece of material releasably attached to the frame with a single pull suture.

The outer skirt can be made from a biocompatible fabric, such as a polyethylene terephthalate (PET) fabric. The fabric can be, for example, a woven, knitted, braided fabric, and/or a non-woven fabric (for example, felt). In other examples, the outer skirt can be made from a non-fabric material, such as a sheet of material made of a polymer (for example, polyurethane), which can be formed, for example, by extruding, molding or otherwise forming a sheet of material from a liquified polymer. In still other examples, the outer skirt can be made from natural tissue, such as pericardial tissue (for example, bovine pericardium, porcine pericardium, or pericardium from other sources).

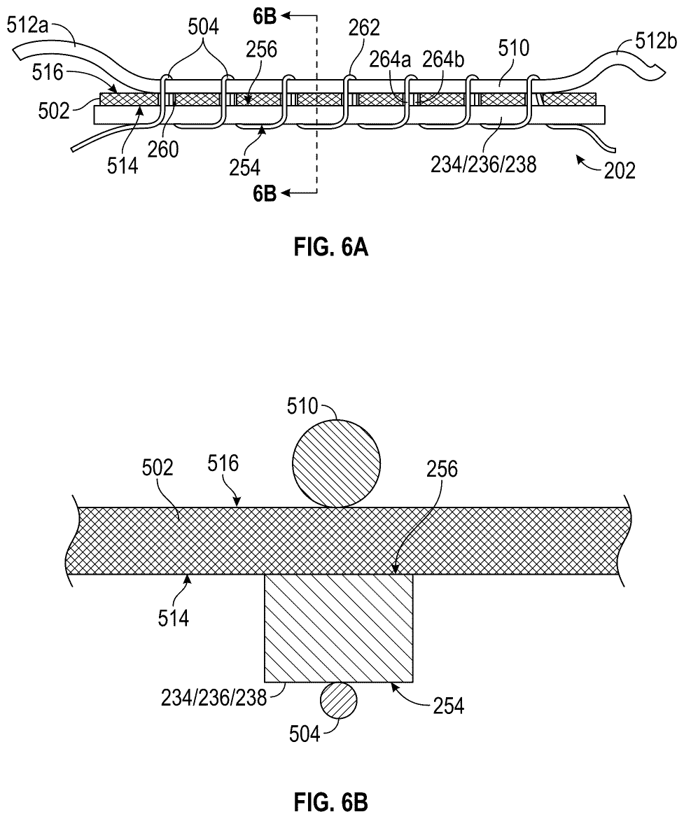

FIG. 6A illustrates an example of a suture line along one of the angled struts (for example, one of the angled struts 234, 236 or 238), and FIG. 6B shows a cross-section of the suture line (taken along the line 6B-6B shown in FIG. 6A). It will be appreciated that the angled strut 234/236/238 is merely exemplary and a similar configuration of a suture line can extend along other struts of a frame (such as, for example, an axial strut 232 or an angled strut 218). As can be seen in FIG.6, in some examples, an interior face 514 of the outer skirt 502 is oriented toward and contacts or is abutted to the exterior surface 256 of the angled strut 234/236/238, and the pull suture 510 contacts or is abutted to an exterior face 516 of the outer skirt 502. The retaining sutures 504 extend around the angled strut 234/236/238, through the material of the outer skirt 502 (that is, being disposed within a plurality of apertures or openings 260 in the material of the outer skirt), and around the pull suture 510 in a whip stitch pattern along the length of the strut 234/236/238. Each whip stitch 262 of the retaining sutures 504 includes a leading end portion 264a that extends from the strut 234/236/238 outwardly through an aperture 260, around the pull suture 510, and transitions to a trailing end portion 264b that extends inwardly through the same opening 260, around the strut, where the trailing end portion 264b transitions to the leading end portion of the next whip stitch 262. In this manner, each whip stitch 262 is threaded through a single opening 260, similar in pattern to a thread in a machine-sewn lock stitch. As can be seen, the pull suture 510 prevents the stitches 262 from being pulled through the openings 260 when the pull suture is positioned along the suture line. Therefore, the outer skirt 502 can be secured to the frame 202 via a force exerted by the retaining sutures 504 on the pull suture 510 and the interior surface 254 of the angled strut 234/236/238 when the pull suture is disposed through the suture line.

In some examples, the apertures or openings 260 in the material of the outer skirt can be pre-formed, such as by laser drilling, cutting, stamping or other suitable techniques known in the art. As best shown in FIG. 8C, the openings 260 can be pre-formed in a skirt portion 502a at spaced apart locations along an undulating path corresponding to the locations of the struts 234, 236, and 238. In other examples, the apertures or openings 260 can be formed as the retaining sutures are stitched through the material of the outer skirt (that is, the trailing end portion 264b of an individual stitch 262 is stitched back through the same opening 260 through which the leading end portion 264a extends).

When the pull suture 510 is removed (via for example, pulling on one of the loose ends 512a or 512b in a direction parallel to the suture line or outwardly from the frame), the retaining sutures 504 are no longer retained by the pull suture 510. Thus, as the outer skirt 502 is pulled away from the frame 202 (or the frame 202 is pulled away from the outer skirt 502), the stitches 262 can be withdrawn through the openings 260 of the outer skirt 502, resulting in the interior face 514 of the outer skirt separating from the angled strut 234/236/238 and the stitches 262 remaining attached to the frame.

In some examples, the interior face 514 of the outer skirt 502 may be comprised of a thromboresistant material or surface that can resist tissue ingrowth and improve ease of separation of the outer skirt 502 from the frame 202. Further, in some examples, the exterior face 516 of the outer skirt 502 may be formed in a manner or comprised of a material that encourages tissue ingrowth or may be comprised of the thromboresistant material.

In some examples that encourage tissue ingrowth, as discussed above, the exterior face 516 of the outer skirt 502 can be the outer surface of a pile or plush layer of a fabric (for example, formed from PET yarns), such as a velour, terrycloth, or a towel. In other words, the fabric used to form the outer skirt can include large number of filaments, such as closed loops (a looped pile), open fringes (a cut pile) or other filament forms, which promote tissue ingrowth into the filaments and sealing of the outer skirt via. The pile layer of the fabric can enhance sealing by mechanically slowing the flow of blood by creating a large micro-scale face area (for example, where v=0 (boundary condition)). Additionally, In some examples, the pile layer can agitate the surrounding blood and/or tissue by exposure thereof to a large surface area, which can stimulate the foreign body response (FBR). Further details of skirts that are configured to promote tissue ingrowth are disclosed in U.S. Pat. No. 11,013,600 and U.S. Patent Publication Nos. 2019/0365530 and 2019/0374337, each of which is incorporated by reference herein.

In some examples that encourage tissue ingrowth, the interior surface and/or the exterior surface of the outer skirt can include a thromboresistant coating or layer. For example, the outer skirt can include a hermetic layer comprising a smooth hydrophobic polymer material with very small pores or no pores. Exemplary materials for the hydrophobic polymer material can include, but are not limited to, polytetrafluoroethylene (PTFE), expanded PTFE (ePTFE), urethane, polyurethane (PU), thermoplastic PU (TPU), silicone, or combinations or copolymers thereof. In one example, the hydrophobic polymer material comprises electrospun urethane layers with ePTFE (such as Bioweb™, sold by Zeus Industrial Products Inc., Orangeburg, SC). In another example, the hydrophobic polymer material comprises a copolymer of silicone and TPU (such as Quadrasil™, sold by Biomerics, Salt Lake City, UT). Other hydrophobic polymer materials or combinations thereof that are not specifically listed above, but are otherwise capable of forming a hermetic later that is substantially nonporous or has sufficiently small size pores that discourage cellular ingrowth, are also possible in some implementations. For example, such materials and hermetic seals that can be used in combination with the exemplary outer skirt and prosthetic valves described herein are disclosed in U.S. Provisional Patent Application No. 63/112,080, which is incorporated by reference herein.

In other examples, the interior surface and/or the exterior surface of the outer skirt can be coated in a thromboresistant material, such as a hydrogel coating, a heparin coating, a silicone coating, or other materials or combinations thereof. Examples of hydrogel coatings that can be used in combination with the exemplary outer skirt and prosthetic valves described herein are disclosed in U.S. Provisional Patent Application No. 63/211,384, which is incorporated by reference herein.

In certain examples, the outer skirt 502 can comprise a pile or plush fabric, such as velour, with a thromboresistant layer applied to the inner surface of the fabric. The pile layer forms the outer surface of the skirt 502 and the thromboresistant layer forms the inner surface of the skirt 502.

It will be appreciated that tissue ingrowth on the exterior face of the outer skirt may enable stable positioning of the prosthetic heart valve while implanted and operating in the native heart valve. It will be further appreciated that use of a thromboresistant material or layer on the interior face of the outer skirt may provide improved separation of the outer skirt from the implantation site and use of a thromboresistant material or layer on the exterior face of the outer skirt may reduce cutting of surrounding tissue during explant.

In some examples, the retaining sutures can be thinner fibers relative to a thicker pull suture(s). In some examples, the retaining sutures can be comprised of a thinner, high tensile strength biocompatible material, such as an ultra-high molecular weight polyethylene (UHMPE) force fiber® or other similar material or combinations thereof. In one specific example, the retaining sutures can be formed from UHMPE force fiber and can be sufficiently thin and resistant to force such that the retaining sutures can cut through overgrown tissue on the outer surface of the outer skirt as the frame is separated from the outer skirt. In some examples, the pull suture can be comprised of a thicker, high tensile strength, biocompatible material, such as a monofilament comprised of polypropylene (for example, Prolene 4-0), polytetrafluoroethylene (PTFE), expanded PTFE (ePTFE), or other similar materials or combinations thereof. In one specific example, the pull suture can be a microfilament comprised of Prolene, which is configured to readily separate from or be pulled away from any surrounding ingrown tissue when pulled on by the surgeon during an explant procedure.

In some examples, the pull suture material can have a distinct coloration or a colored coating (for example, a green, black, or blue coloration or coating) and/or can be otherwise tagged such that it can be identified by a surgeon during an explant procedure. In some examples, an entire length of the pull suture can, for example, be comprised of a brightly colored material or have a brightly colored coating. In other examples, a portion of the pull suture (for example, a loose end portion of the pull suture) can be comprised of a colored material, have a brightly colored coating, and/or can include a brightly colored tag or flap attached thereto. In other examples, the pull suture or a portion thereof can include a material, coating, or tag that can be visualized using a specialized visualization apparatus. For example, the pull suture can comprise a radiopaque substance or one or more radiopaque markers that can be visualized via a fluoroscopy or x-ray device. For example, the one or more radiopaque markers can be embedded within or attached to an outer surface of the pull suture.

### Exemplary Methods

As discussed above, the configuration of the prosthetic heart valve 500 may be particularly advantageous for implant and/or explant procedures, such as those discussed below.

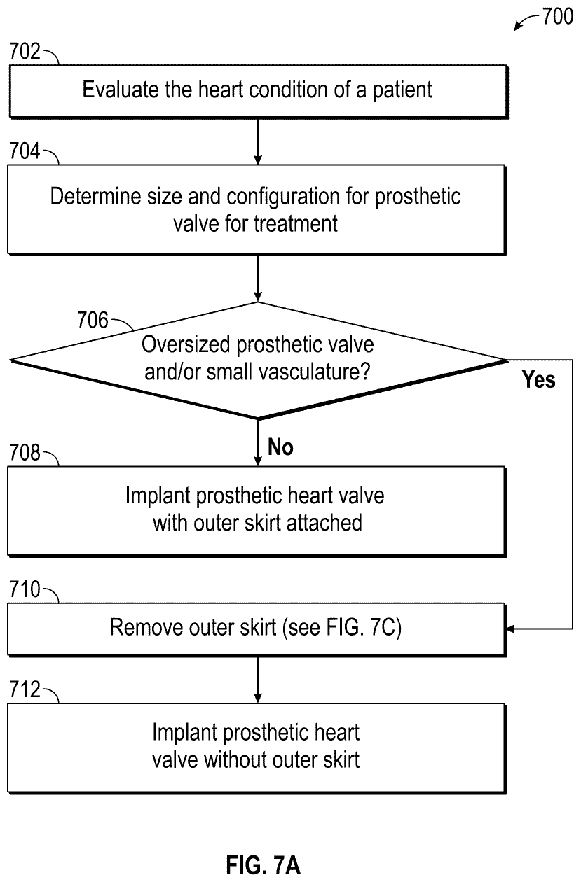

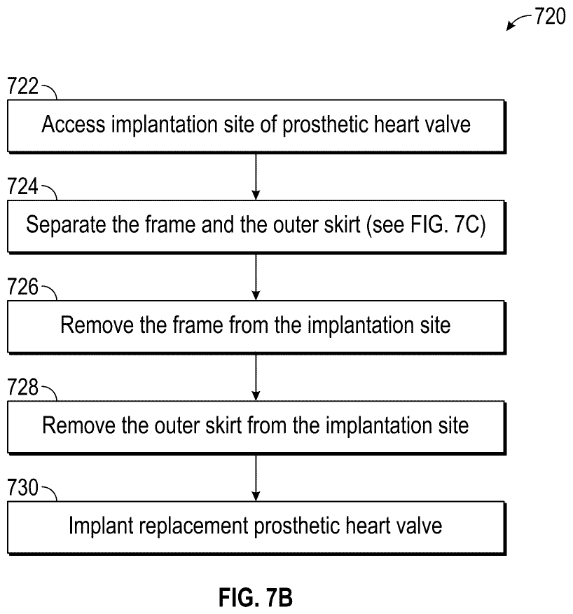

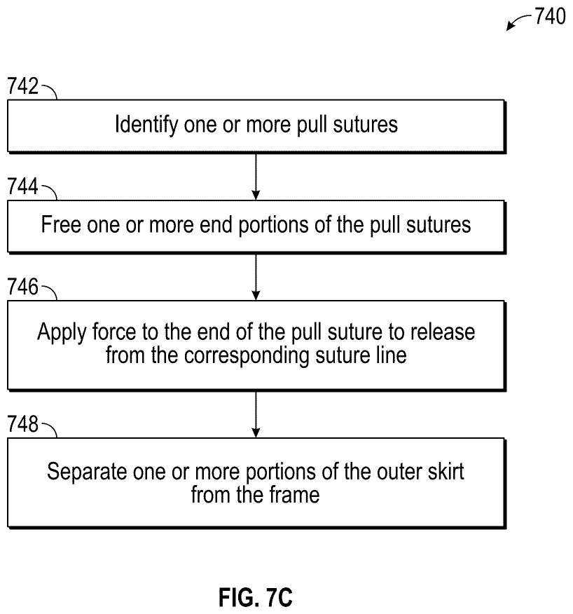

An exemplary method of implant 700 is shown in the flow diagram of FIG. 7A. First, at step 702, a physician can evaluate the heart condition of a specific patient who will have a prosthetic valve implanted within his or her heart via transcatheter implantation. For example, prior to an implantation procedure, imaging or other analysis techniques can be performed on the patient to determine a type of heart condition (for example, aortic stenosis or aortic insufficiency), as well as anatomy of the heart (such as, for example, a size of the native annulus) and vasculature of the patient, such as echocardiography, electrocardiogram, x-ray, cardiac MRI, cardiac catheterization, and/or other imaging or assessment modalities. Based on such analyses, at step 704, the physician may determine an appropriate size and/or configuration for a prosthetic valve for treatment of the patient. For example, at step 706, a physician can determine whether vasculature of the patient through which the prosthetic valve will be delivered is particularly small or narrow, or whether the patient requires or would benefit from an oversized prosthetic valve for treatment (for example, for treatment of AI). Alternatively, the physician can determine whether the patient requires a regularly sized prosthetic valve and/or a prosthetic valve that includes an outer skirt (for example, for treatment of AS).

If, for example, the patient does not have small vasculature and/or does not require an oversized prosthetic valve, the physician may leave the outer skirt attached to the frame, and implant the prosthetic valve via transcatheter techniques (step 708). If, however, the patient does have small vasculature and/or requires an oversized prosthetic valve, the p can first separate the outer skirt from the frame of the prosthetic valve (step 710, and described below with reference to FIG. 7C), thereby removing the outer skirt prior to implantation of the prosthetic valve. Next, the prosthetic valve with the outer skirt removed can be implanted via transcatheter techniques (step 712).

Turning to FIG. 7B, an exemplary method of explant 720 of a prosthetic valve having a removeable skirt is shown and described. First, as indicated at step 722, the site of implantation of a prosthetic heart valve and a detachable outer skirt (such as, for example, the prosthetic heart valve 500) is accessed by a surgeon. In certain examples, the implantation site can be accessed via surgical procedures that provide direct access to the heart, such as open-heart or partial open-heart surgical techniques. In other examples, the implantation site can be accessed via minimally invasive procedures, such as transcatheter (for example, transarterial or trasfermoral) surgical techniques that do not require opening the chest cavity and placing the patient on a cardiopulmonary bypass machine.

Per step 724, after accessing the implantation site, the surgeon can separate the frame from the outer skirt (described below with reference to FIG. 7C). After full separation from the outer skirt, per step 726, the frame can be removed from the implantation site. In some examples, the frame can be folded or radially compressed to reduce its size prior to removal from the implantation site, such as removal via a catheter. The catheter can include one or more grasping mechanisms for grasping the frame and pulling it into a sheath of the catheter, which effectively radially collapses the frame as it is pulled into the sheath. Thereafter, the catheter along with the prosthetic valve 500 (without the outer skirt) can be removed from the patient's body. In other examples, such as in open-heart explant procedures, the frame can be removed with or without folding or compressing, and a need or folding or compressing may depend on the anatomy of the patient, the location of the prosthetic valve/implantation site, and/or the specific type of procedure.

Per step 728, the outer skirt can then be removed from the implantation site. In some cases, the outer skirt may remain attached to the implantation site if tissue is ingrown therein. In such situations, the portions of the outer skirt and/or the native leaflets can be excised or cut from the implantation site along the native scallop line, which can be performed without other components of the valve interfering or limiting access to the region. In other words, removal of the frame prior to removal of the outer skirt may provide increased access to the implantation site and easier cutting of the outer skirt and/or native leaflets from the implantation site relative to a prosthetic valve having an outer skirt that is non-detachable (for example, a prosthetic valve that includes an outer skirt sutured directly to the frame in a manner that cannot be easily released). In other situations, the outer skirt may have little or no tissue ingrowth and be easily separable from the implantation site.

Lastly, at step 730, a new prosthetic valve can be implanted at the implantation site via, for example, the implantation method discussed above with reference to FIG. 2. The same catheter used to remove the prosthetic valve 500 or a separate delivery catheter (for example, delivery catheter 100) carrying a replacement prosthetic valve can be used to position and deploy the replacement prosthetic valve within the native heart valve. Alternatively, if the patient's chest cavity is opened, a surgical prosthetic heart valve can be implanted using known surgical techniques.

FIG. 7C shows an exemplary method 740 for separation of an outer skirt from a frame of a prosthetic valve and is described with respect to the corresponding illustrations shown in FIGS. 8A-8D. Per step 742, the medical practitioner or surgeon can identify one or more pull sutures on the prosthetic valve. In certain examples, as discussed above, a pull suture (or a portion thereof) can have a distinct coloration or coating, or a colored tag that enables the surgeon to directly visually identify the pull suture on the outer surface of the prosthetic heart valve, such as, for example, during preparation of the prosthetic valve prior to implantation thereof, or, in other examples, during an open-heart explant procedure or a transcatheter explant procedure utilizing a camera. In other examples of transcatheter explant, a pull suture (or a portion thereof) can include a radiopaque substance or markers that the surgeon can visualize utilizing a detection and visualization apparatus, such as a fluoroscopy or x-ray device. In any of the foregoing examples, the prosthetic valve may have a single pull suture extending along a suture line of the outer skirt (comprising one or more outer skirt portions) or two or more pull sutures which extend along the suture line of portions or sections of the outer skirt. Therefore, the surgeon may identify a location of each pull suture. Further, the surgeon may in some examples identify an entirety of the pull suture, or, in other examples, the surgeon may identify only a portion of the pull suture, such as an end thereof that is attached to the frame or another portion of the prosthetic valve.

Next, at step 744, the surgeon can free the end(s) of the identified pull suture(s). As discussed above, the ends of the pull sutures can extend beyond the suture line of the outer skirt and be releasably attached to a portion of the prosthetic valve (for example, the frame, one or more leaflets, an inner skirt, or the outer skirt) in a manner than enables a position of the ends to be retained while the prosthetic valve is implanted and further enables the surgeon to easily free the ends during preparation of the prosthetic valve for implant or during an explant procedure, such as via cutting or pulling on the pull suture proximate to the attachment sites of the ends.

In some examples, the ends can be attached to a portion of the prosthetic valve via an adhesive. In other examples, the ends can be secured to one of the frame struts via being at least partially disposed beneath a suture or an elastomeric member covering the strut. In still other examples, the ends of the pull suture(s) may be loosely sutured to the outer skirt with one or more stitches (for example, in-and-out stitches) and a portion of the pull suture adjacent to the end can be pulled to free the end from the loose sutures. In other examples, the ends of the pull suture(s) can be tied off to a strut of the frame. The surgeon can cut the ends of the pull suture close to the frame in order to disconnect the pull suture from the frame. In some examples, the ends of the pull suture can be tied off or otherwise attached to the frame at the outflow end of the frame, or at least along the outflow portion of the frame downstream of the outer skirt, which typically is positioned within the aorta just above aortic annulus. Securing the ends of the pull suture along the outflow portion of the frame therefore can allow greater access to the ends of the pull suture during an explant procedure.

As can be seen in the exemplary outer skirt removal illustration of FIG. 8A, the ends 512 of the pull suture 510 have been freed from their attachment to the prosthetic valve 500 and are accessible to the surgeon. A remaining portion of the pull suture 510 is disposed along the suture line and captured between the retaining sutures 504 and the exterior face 516 of the outer skirt portion 502a such that the outer skirt portion 502a is attached to the frame 202. In some examples where the surgeon has direct access to the prosthetic valve, such as during preparation for implant or open-heart explant, the scissors or another cutting device can be used to free the ends 512. In other examples, if a transcatheter or percutaneous explant procedure is performed, the physician can insert an elongated catheter having a cutting blade at a distal end thereof into the patient's vasculature and advance the catheter to the prosthetic valve 500. The physician can manipulate the catheter to cut the ends 512 of the pull suture 510 from their attachment to the prosthetic valve using the cutting blade.

Returning to FIG. 7C, at step 746, the surgeon can grasp one of the freed ends of the pull suture (via by hand or by a tool) and apply a pulling force to the pull suture to withdraw the pull suture from the retaining sutures along the suture line. For example, FIG. 8B illustrates the prosthetic valve 500 in a state where the pull suture is fully removed and the outer skirt 502a is loosely attached to the frame 202 by the retaining sutures 504 extending through the openings 260 in the outer skirt. If the procedure is performed percutaneously, the catheter can include a grasping mechanism for grasping and pulling the end 512 of the pull suture.

As discussed above with reference to FIGS. 6A and 6B, the stitches 262 of retaining sutures 504 can each extend through respective openings 260 in the outer skirt 502 and can be withdrawn therethrough after the pull suture 510 is removed. Accordingly, as indicated at step 748, subsequent to the removal of the pull suture(s), the one or more portions of the outer skirt can be separated from the frame. In prosthetic valve examples including more than one outer skirt portion (for example, three skirt portions), each one can be released via its respective pull sutures for step-wise separation of portions of the frame from the outer skirt.

In some examples, the frame can be grasped (by hand or via a tool) by the surgeon and pulled away from the outer skirt, thereby causing the stitches 262 to be pulled through the apertures 260 in the outer skirt. As discussed above, the retaining sutures can be comprised of a thin, high tensile strength material, and the pulling of the stitches through the outer skirt and away from the implantation site may cause the stitches to cut through any overgrown tissue. FIG. 8C illustrates an exemplary stage of an explant procedure where the frame 202 is partially separated from the outer skirt portion 502a as the frame is pulled away therefrom. Accordingly, a portion of the retaining sutures 504 still extend through the outer skirt 502, while another portion of the retaining sutures (not shown) have been pulled through the outer skirt 502.

FIG. 8D illustrates another exemplary stage of the separation procedure where the frame 202 is fully separated from the outer skirt, and the retaining sutures 504 remain attached to the angled struts 234, 236, and 238 of the frame 202.

It will be appreciated that the prosthetic heart valves disclosed herein can be made via the disclosed examples of methods of manufacture that enable releasable attachment of one or more portions of an outer skirt to a frame. In some examples, a method of manufacture can include: obtaining a frame comprising interconnected struts arranged between an inflow end and an outflow end of the frame; overlaying one or more portions of the detachable outer skirt on an exterior surface of the frame; and forming on or more sutures lines on the outer skirt by looping at least one retaining suture around specified ones of the struts, through the outer skirt such that each stitch of the retaining suture extends through a single opening in the skirt, and over one or more pull sutures on an exterior surface of the outer skirt.

In some examples, the method can further include forming a releasable attachment between the ends of the one or more pull sutures and an attachment region on the outer skirt, the frame, and/or a valvular structure disposed on an interior of the frame. In some examples, the forming the temporary attachment can include capturing the end within the attachment region via at least one of an adhesive, a suture, an elastic band or tying the end to the frame. In some examples, the overlaying the one or more portions of the outer skirt on the exterior surface of the frame includes respectively aligning each of a first portion, a second portion, and a third portion of the outer skirt with a cusp edge of a first leaflet, a cusp edge of a second leaflet, and a cusp edge a third leaflet of a valvular structure, the valvular structure attached to an interior of the frame. In some examples, the overlaying the one or more portions of the outer skirt on the exterior surface of the frame includes overlapping adjacent edges of the one or more portions of the outer skirt. In some examples, the forming the one or more suture lines includes forming a suture line on each of the overlapping edges of the one or more portions of the outer skirt.

### Additional Examples of the Disclosed Technology

As discussed above, a prosthetic valve can include the frame 202 or a different frame. Additional examples of frames that can be utilized in a prosthetic valve having a detachable outer skirt are illustrated in FIGS. 9A-9F.

Referring to FIG. 9A, a prosthetic heart valve assembly 900 comprises a valve component 902 and an anchoring frame 904 coupled to a valve frame 906 of the prosthetic heart valve 902. Due to their relative locations, the anchoring frame 904 can also be referred to as “the outer frame,” and the valve frame 906 can also be referred to as “the inner frame.” Although not specifically illustrated in FIGS. 9A-9F, a detachable outer skirt, such as the outer skirt 502 can be releasably attached to the anchoring frame 904 and/or the valve frame 906 on an outer surface of the prosthetic heart valve assembly 900.

The prosthetic heart valve assembly 900 can be radially compressed (which can also be referred to as “crimped”) to a delivery configuration (with or without an outer skirt attached thereto) and advanced through a patient's vasculature to an implantation location.The prosthetic heart valve assembly 900 can be radially expanded from the delivery configuration to a functional configuration and positioned in a native heart valve annulus. The valve component 902 is configured for regulating the flow of blood in one direction through the prosthetic heart valve assembly 900. The anchoring frame 904 comprises a plurality of tissue-engagement elements (for example, the projections 924). In alternate examples, the anchoring frame can be eliminated from the prosthetic heart valve assembly 900 and the valve frame 906 can include a plurality of tissue-engagement elements (for example, projections similar in configuration of the projections 924).

In some examples where the outer skirt is removed prior to delivery of the prosthetic heart valve assembly 900, such as for treatment of AI patients, the projections 924 are configured to help secure the prosthetic heart valve 902 to native heart valve tissue and/or to help promote tissue ingrowth between the native tissue and the prosthetic heart valve assembly 900. Alternatively, the outer skirt can remain attached on the outer surface of the prosthetic heart valve assembly 900 and cover all or a portion of the anchoring frame 904, such that the native heart valve tissue is shielded from the projections 924. For example, the outer skirt can remain attached to the prosthetic heart valve assembly 900 for treatment of AS patients, and can fill in gaps in calcified tissue to create a seal between the implanted prosthetic valve and the native annulus (as described above).

Referring again to FIG. 9A, the valve component 902 comprises a valvular structure 908, and optionally includes one or more interior sealing members 910 (which can also be referred to as “a sealing skirt” or “a PVL skirt”). The sealing member 910 can be formed of various materials such as a fabric or cloth. In some instances, the sealing member can be formed from polyethylene terephthalate (“PET”) and/or ultra-high molecular weight polyethylene (“UHMWPE”) fabric. In other examples, various other synthetic or natural materials can be used.

The valve frame 906 is configured for supporting the valvular structure 908 and/or to help secure the prosthetic heart valve assembly 900 to native heart valve tissue (for example, a native heart valve annulus and/or native leaflets). The valvular structure 908 comprises one or more leaflets 919 configured to open to allow blood flow through the valve component 902 from an inflow end portion 912 to an outflow end portion 914. The leaflets 918 are also configured to close to prevent or restrict blood flow through the valve component 902 from the outflow end portion 914 to the inflow end portion 912. The sealing member 910 is configured for reducing or eliminating blood flow around the valvular structure 908 and/or native tissue (which can also be referred to as “paravalvular leakage,”“perivalvular leakage”, or “PVL”).

FIGS. 9C and 9E depict the valve frame 906 with the other components removed. FIG. 9C depicts the valve frame 906 in an annular configuration, which corresponds to its functional configuration, and FIG. 9E depicts the valve frame 906 in a flat configuration for purposes of illustration. The valve frame 906 comprises a plurality of interconnected struts. In some examples, the struts form a plurality of cells. For example, referring to FIG. 9E, the struts of the valve frame 906 form a plurality of rows of closed cells, including a first row of closed cells I, a second row of closed cells II, a third row of closed cells III, and a fourth row of closed cells IV. In the illustrated example, the cells of row I are larger than the cells of rows II and III but are smaller than the cells of row IV. The cells of row II are the same size or at least substantially the same size as the cells of row III. In the depicted example, the cells are generally hexagonal shaped. In other examples, a valve frame can comprise various other numbers of rows of cells, the cells can comprise different sizes, and/or the cells can comprise different shapes.

The valve frame 906 also comprises a plurality of commissure windows 916 (for example, three in the illustrated example). The commissure windows 916 are configured for coupling the commissures of the leaflets 918 to the valve frame 906.

Similar to the frames 12 and 202, the valve frame 906 can be made of any of various suitable plastically-expandable materials (for example, stainless steel, etc.) and/or self-expanding materials (for example, Nitinol). When the valve frame comprises plastically-expandable material, the valve frame (and thus prosthetic heart valve assembly) can be crimped to a radially compressed state on a delivery catheter and then expanded inside a patient by an inflatable balloon or equivalent expansion mechanism of a delivery apparatus. When the valve frame comprises self-expandable material, the valve frame (and thus the prosthetic heart valve assembly) can be crimped to a radially compressed state and restrained in the compressed state by a sheath or equivalent mechanism of a delivery apparatus. Once inside the body, the prosthetic heart valve assembly can be advanced from the delivery sheath, which allows the valve to self-expand to its functional size.

Suitable plastically-expandable materials that can be used to form the frames disclosed herein (such as, for example, the valve frame 906) include, metal alloys, polymers, or combinations thereof. Example metal alloys can comprise one or more of the following: nickel, cobalt, chromium, molybdenum, titanium, or other biocompatible metal. In some examples, the valve frame 906 can comprise stainless steel. In some examples, the valve frame 906 can comprise cobalt-chromium. In some examples, the valve frame 906 can comprise nickel-cobalt-chromium. In some examples, the valve frame 906 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

Additional details about valve frames, valve structures, outer skirts, and the manner in which a valve structure and/or an outer skirt can be secured to a valve frame can be found in U.S. Pat. No. 6,730,118, which is incorporated by reference herein, and well as U.S. Pat. Nos. 9,393,110 and 11,096,781, previously incorporated herein.

Referring to FIG. 9D, the anchoring frame 904 comprises a plurality of struts configured in an annular shape. As depicted in FIG. 9B, the anchoring frame 904 also comprises a plurality of tissue-engaging elements. In the illustrated example, the tissue-engaging elements are projections 924 (which can also be referred to as “anchors”) extending from the struts of the anchoring frame 904. The projections 924 are configured to engage (and in some instances penetrate and embed within) the native heart valve tissue. In some examples, the projections 924 can be in the form of barbs and/or can have pointed ends. In this manner, the projections 924 can increase the frictional engagement between the prosthetic heart valve assembly 900 and native heart valve tissue (if the outer skirt is removed prior to delivery of the prosthetic valve), which can help to reduce migration of the prosthetic heart valve assembly 900 relative to the native heart valve tissue after it is released from the delivery apparatus. The projections can also help to improve tissue ingrowth and/or reduce PVL.

The projections 924 can extend in various directions from the struts of the anchoring frame 904. For example, in the illustrated example, some of the projections 924 extend from the struts at an angle relative to a central longitudinal axis extending from the inflow end to the outflow end of the prosthetic heart valve assembly. In some instances, the projections are perpendicular or at least substantially perpendicular (for example, forming an angle of 80-100 degrees) to the struts from which they extend. In other examples, the projections can extend from their respective struts at various other angles (for example, between 1-79 degrees). For example, in some instances, the projections can extend from their respective struts at an angle of about 45 degrees such that projections are parallel or at least substantially parallel to a central longitudinal axis extending from the inflow end to the outflow end of the prosthetic heart valve assembly.

The projections can comprise various shapes and lengths such that the projections provide sufficient retention force for the prosthetic heart valve assembly, while reducing potential harm to the surrounding tissue. For example, in the illustrated example, the projections comprise tines or spikes. In other examples, the projections can comprise ball-shaped bulges and/or a rectangular shape. Additionally or alternatively, one or more of the projections can comprise a curved shape, a hook shape, a cross shape, a T-shape, and/or a barbed shape. Various combinations of shapes and/or sizes of projections can be used.

The anchoring frame 904 of the prosthetic heart valve assembly 900 can be formed as a separate component that is attached to the valve frame 906 to form the assembly. FIGS. 9D and 9F depict the anchoring frame 904 of the prosthetic heart valve assembly 900 detached from the valve frame 906. FIG. 9D depicts the anchoring frame 904 in an annular configuration, and FIG. 9F depicts the anchoring frame 904 in a flat configuration.

In some examples, the struts of the anchoring frame 904 can form a plurality of cells. For example, referring to FIG. 9F (which depicts the anchoring frame 904 in a flat configuration), the struts of anchoring frame 904 form a plurality of rows of closed cells, including a first row of closed cells I, a second row of closed cells II, and a third row of closed cells III. In the illustrated example, the cells of the first row I are larger than the cells of rows II and III. The cells of the second row II are the same size or at least substantially the same size as the cells of the third row III. The cells are generally hexagonal shaped. In other examples, an anchoring frame can comprise various other numbers of rows of cells (for example, 1, 2, 4), the cells can comprise different sizes, and/or the cells can comprise different shapes.

The anchoring frame 904 is configured such that the struts and the cells of the rows I, II, and III of the anchoring frame 904 align with the struts and the cells of the rows I, II, and III of the valve frame 106, respectively (see, for example, FIG. 9A). In this manner, strut junctions 926 of the anchoring frame 904 can be coupled to strut junctions 928 of valve frame 906, as depicted in FIG. 9B. In the illustrated example, the anchoring frame 904 is coupled to the valve frame 906 with sutures 930. In some examples, the anchoring frame 904 comprises openings 932, which can be configured to receive the sutures 930. It should be noted that, for purposes of illustration, the sutures are not shown in FIG. 9A. In other examples, the anchoring frame can be coupled to the valve frame in various other ways (for example, fasteners, welding, adhesive, etc.). By coupling the junctions 926 of the anchoring frame 904 to junctions 928 of the valve frame 906, the anchoring frame 904 can, for example, expand and/or or compress simultaneously with the valve frame 906.

In some examples, the anchoring frame 904 is removably coupled to the valve frame 906 (for example, with the sutures 930 and/or fasteners). As used herein, “removably coupled” means coupled in such a way that two components are coupled together and can be separated without plastically deforming either of the components. In other examples, the anchoring frame can be permanently coupled to the valve frame (for example, via welding). As used herein, “permanently coupled” means coupled in such a way that the two components cannot be separated without plastically deforming at least one of the components.

The anchoring frame can be made of any of various suitable plastically-expandable materials (for example, stainless steel, etc.) and/or self-expanding materials (for example, Nitinol). When the anchoring frame comprises plastically-expandable material, the anchoring frame (and thus prosthetic heart valve assembly) can be crimped to a radially compressed state on a delivery catheter and then expanded inside a patient by an inflatable balloon or equivalent expansion mechanism of a delivery apparatus. When the anchoring frame comprises self-expandable material, the anchoring frame (and thus the prosthetic heart valve assembly) can be crimped to a radially compressed state and restrained in the compressed state by a sheath or equivalent mechanism of a delivery apparatus. Once inside the body, the prosthetic heart valve assembly can be advanced from the delivery sheath, which allows the prosthetic heart valve assembly to expand to its functional size.

Suitable plastically-expandable materials that can be used to form the frames (such as, for example, the anchoring frame 904) disclosed herein include, metal alloys, polymers, or combinations thereof. Example metal alloys can comprise one or more of the following: nickel, cobalt, chromium, molybdenum, titanium, or other biocompatible metal. In some examples, the anchoring frame 904 can comprise stainless steel. In some examples, the anchoring frame 904 can comprise cobalt-chromium. In some examples, the anchoring frame 904 can comprise nickel-cobalt-chromium. In some examples, the anchoring frame 904 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

In other examples, the anchoring frame 904 can be omitted and anchoring features (for example, projections 924) can be formed on one or more of struts of the valve frame 906. In one specific example, the projections 924 can be formed on the struts forming the cells in rows I, II, and III in the same manner as shown in FIG. 9F for the anchoring frame 904.

Additional description and examples of prosthetic valves comprising frames having anchoring features for improving the anchoring of the prosthetic valve in an AI patient are disclosed in PCT Application No. PCT/US2021/034399, filed May 27, 2021, which is incorporated herein by reference. Examples of prosthetic valves according to the present disclosure can comprise any of the prosthetic valves disclosed in PCT Application No. PCT/US2021/034399 and a removable outer skirt (for example, outer skirt 502) detachably connected to the frame as disclosed herein.

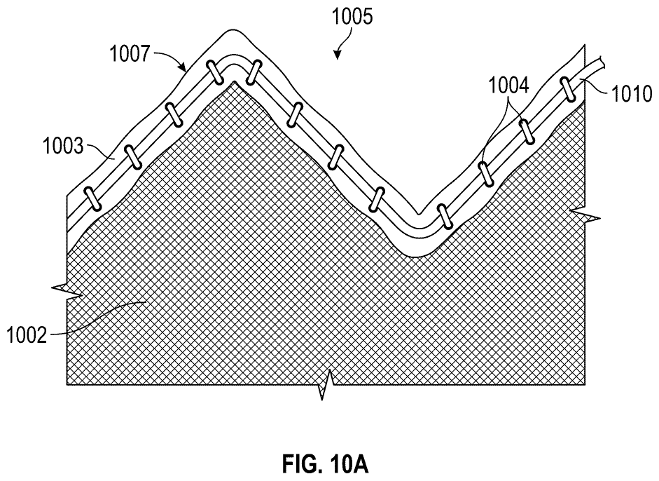

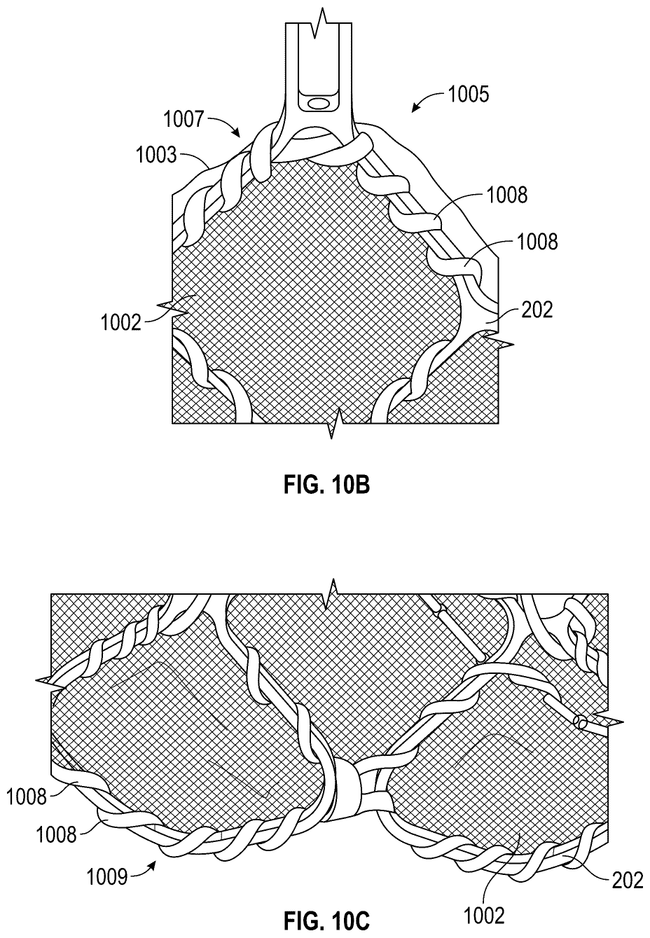

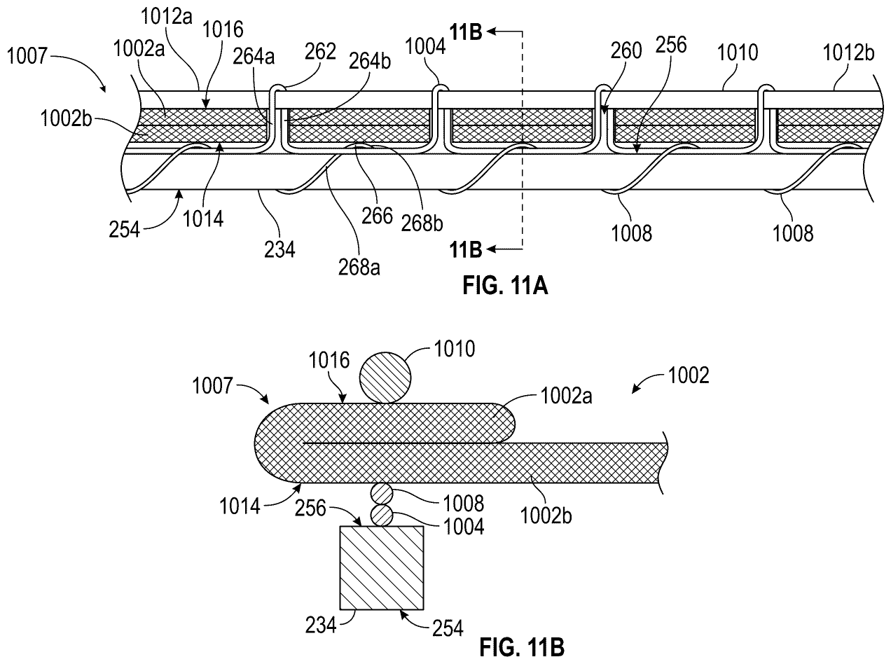

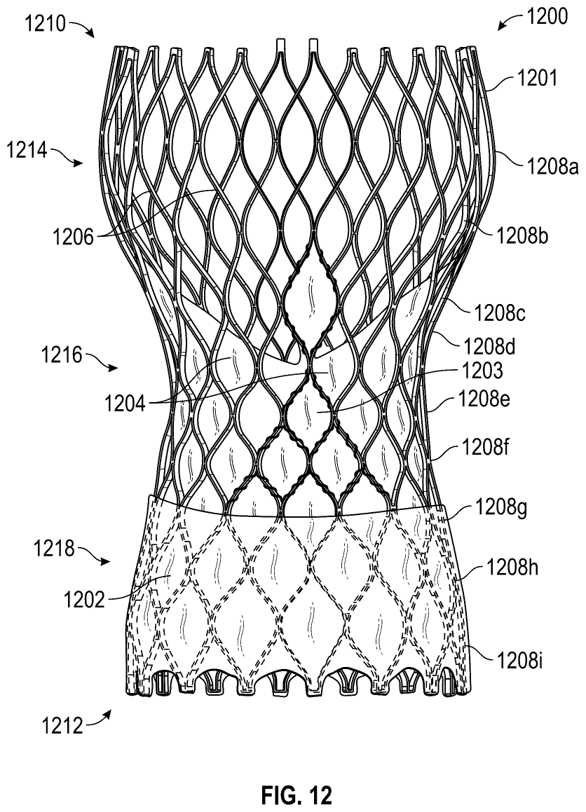

In other examples, a prosthetic valve including the frame 202 (or a different frame, such as a frame 1201 discussed below) can a different configuration or type of detachable outer skirt. Additional examples of detachable outer skirts that can be utilized in prosthetic valves are illustrated in FIGS. 10A-11, as well as FIGS. 12-15.

Referring to FIG. 10A-11, in some examples, the outer skirt can include a fused or molten edge at one or both of the inflow edge or the outflow edge of the outer skirt. The fused or molten edges may be a region where fibers of the outer skirt are melted and/or fused together to prevent the fibers of the outer skirt material from unraveling and to maintain the woven configuration of the fibers. The fused edges, however, may be abrasive and may damage the leaflet material (such as for example, the leaflets 204) of the valvular structure disposed on the interior of the frame. For example, the leaflet material may be damaged by the fused edge during systole. Accordingly, the fused edges can be oriented away from the leaflets toward the exterior of the prosthetic valve and can be folded over on the exterior surface of the valve to prevent or limit contact between the fused edges and the leaflets of the prosthetic valve. In some examples, the outer skirt can be releasably sutured to the frame along the fold to secure the position of the fused edges and to enable release of the outer skirt from the frame during, for example, explant of the prosthetic valve. It will be appreciated the outer skirt including the fused edges can be made from a biocompatible fabric, such as a polyethylene terephthalate (PET) fabric. The fabric can be, for example, a woven, knitted, and/or braided fabric. In still other examples, the outer skirt can be made from natural tissue, such as pericardial tissue (for example, bovine pericardium, porcine pericardium, or pericardium from other sources).

For example, FIG. 10A shows an exterior view of a section of an outer skirt 1002 including a fused edge 1003 at an outflow end 1005 of the outer skirt that is formed into a folded configuration 1007 and connected to or attached to a frame along a suture line including a plurality of retaining sutures 1004 and a pull suture 1010 (which can be one or more pull sutures that each a at least one free end for removal thereof from the suture line). For illustrative purposes, a frame (such as for example, the frame 202) to which the outer skirt 1002 is attached is not shown in FIG. 10A. However, it will be appreciated that a position of the suture line corresponds to a location of struts of the frame to which the outer skirt 1002 is releasably attached.

In the illustrated example, the outflow end 1005 can have an undulating shape (for example, zig-zag or saw-tooth shape forming a series of triangular projections as shown) that corresponds of the shape of a row of angled struts of the frame, similar to skirt 18 of FIG. 1. The height of the skirt 1002 can vary. Accordingly, the outflow end 1005 can be connected to a selected row of struts of the frame by the suture line, such as the second, third, fourth, etc. row of struts, depending on the number of rows of struts of the frame. In some examples, the frame 202 has the configuration shown in FIGS. 3 or FIG. 5A and has four rows of struts, and the outflow edge 1003 is connected to the third row of struts (the row immediately upstream the commissure attachment posts 242).

FIGS. 10B and 10C illustrate interior views of the outer skirt 1002 attached to the frame 202. Specifically, FIG. 10B shows an interior view of the outflow end 1005 of the outer skirt 1002, and FIG. 10C shows an interior view of an inflow end 1009 of the outer skirt 1002. In the present example, the inflow end 1009 of the outer skirt 1002 does not include a fold, and instead the skirt material may be at least partially wrapped around the struts forming a first row of struts at the inflow end of the frame 202. In other examples, the outer skirt 1002 can include an additional fold at the inflow end, similar to fold 1007, with the inner and outer layers of the fold being secured to struts at the inflow end of the frame. In either example, the inflow end 1009 of the outer skirt 1002 can be releasably attached to the frame by a plurality of retaining sutures and one or more pull sutures (not shown).

In some examples, the suture line at the outflow end 1005 and the inflow end 1009 of the outer skirt 1002 can have a configuration similar to that described above with respect to FIGS. 6 and 6B. In other examples, the suture lines can have a different configuration.

For example, a suture line can include an exemplary non-penetrating technique for coupling the outer skirt to at least one strut of a frame, such as the techniques disclosed in PCT Patent Application US2021/041002, which in incorporated by reference herein. As discussed therein, a cusp end portion of a leaflet, with a primary suture threaded there-through in an in-and-out pattern, can be aligned with a strut such that the cusp end portion is positioned radially inward and optionally in contact with the inner side or surface of the strut of the frame. According to some examples, a secondary suture can be stitched through a primary suture and around at least one section of at least one strut. It will be appreciated that similar stitching patterns and techniques can be utilized for attachment of an outer skirt to a frame, such as for realeasable attachment of the outer skirts disclosed herein.

For example, FIGS. 11A and 11B show one exemplary technique for coupling the outflow end 1005 of the outer skirt 1002 to a frame. Specifically, FIG. 11A illustrates an example of a suture line along one of the angled struts 234, which is similar to the technique of FIGS. 6A and 6B in that an interior face or surface 1014 of the outer skirt 1002 is oriented toward to the exterior surface 256 of the angled strut 234, and the pull suture 1010 contacts or is abutted to an exterior face 1016 of the outer skirt 1002. It will be appreciated that the angled strut 234 is merely exemplary and a similar configuration of a suture line can extend along other struts of a frame (such as, for example, an axial strut 232 or an angled strut 218, 236, or 238).

FIG. 11B shows a cross-section of the suture line (taken along the line 11B-11B shown in FIG. 11A). In the present example, the outflow end 1005 has the folded configuration 1007 and therefore the outer skirt 1002 can include an outer (folded over) layer 1002a and an inner layer 1002b. Further, the retaining sutures 1004 can extend through the material layers 1002a and 1002b of the outer skirt 1002 (for example, the retaining sutures are disposed within a plurality of apertures or opening 260 in the material layers 1002a and 1002b of the outer skirt). Further, each whip stitch 262 of the retaining sutures 1004 can include a leading end portion 264a that extends outwardly through an aperture 260 (in both layers 1002a, 1002b), around the pull suture 1010, and transitions to a trailing end portion 264b that extends inwardly through the same aperture 260 (in both layers 1002a, 1002b), where the trailing end portion 264b transitions to the leading end portion of the next whip stitch 262. In this manner, each whip stitch 262 is threaded through a single opening or aperture 260, similar in pattern to a thread in a machine-sewn lock stitch. As can be seen, the pull suture 1010 prevents the stitches 262 from being pulled through the openings 260 when the pull suture is positioned along the suture line.

As discussed above, in some examples, the apertures or openings 260 in the material layers 1002a and 1002b of the outer skirt can be pre-formed, such as by laser drilling, cutting, stamping or other suitable techniques known in the art, and the openings 260 can be pre-formed in the layers 1002a and 1002b of the outer skirt 1002 at spaced apart locations along an undulating path corresponding to the locations of the struts 234, 236, or 238. In other examples, the apertures or openings 260 can be formed as the retaining sutures are stitched through the material layers 1002a and 1002b of the outer skirt (that is, the trailing end portion 264b of an individual stitch 262 is stitched back through the same opening 260 through which the leading end portion 264a extends).

Differently from the example of FIGS. 6A and 6B, the present example further includes at least one suture forming a series of non-penetrating whip stitches 1008, which can be identical or similar to the configurations described in PCT Patent Application US2021/041002, previously incorporated herein. As can be seen in FIG. 11A, the non-penetrating whip stitches 1008 are formed by passing a leading end portion 268a of a secondary stitch 266 over the retaining sutures 1004 (that is, between the retaining suture 1004 and interior face 1014 of the outer skirt 1002) and then folding a trailing end portion 268b of the secondary stitch 266 back toward the frame and around interior face 254 of the strut 234/236/238 of the frame. Ends of the suture forming the non-penetrating whip stitches 1008 can be secured to the frame at a junction between two or more of the struts via a knot, such as the knots disclosed in PCT Patent Application US2021/041002, previously incorporated herein, or via other configurations of knots, or via other techniques (for example, utilizing an adhesive).

Thus, in this example, the non-penetrating whip stitches 1008 can be secured directly to the strut and the retaining sutures 1004 can be indirectly secured to the strut via the non-penetrating whip stitches 1008. Further, the outer skirt 1002 can be secured to the frame 202 via a force exerted by the retaining sutures 1004 on the pull suture 1010 and the non-penetrating whip stitches 1008 when the pull suture is disposed through the suture line.

When the pull suture 1010 is removed (via for example, pulling on one of the loose ends 1012a or 1012b in a direction parallel to the suture line or outwardly from the frame), the retaining sutures 1004 are no longer retained by the pull suture 1010. Thus, as the outer skirt 1002 is pulled away from the frame 202 (or the frame 202 is pulled away from the outer skirt 1002), the stitches 262 can be withdrawn through the openings 260 within the material layers 1002a and 1002b the outer skirt 1002, resulting in the interior face 1014 of the outer skirt separating from the angled strut 234 (or from an angled strut 236 or 238) and the stitches 262 and the second secondary stitches 266 remaining attached to the frame.

Additional methods of forming the stitch pattern, knotting the ends of the sutures, and other stitching techniques that can be utilized in combination with the prosthetic valves and detachable outer skirts disclosed herein are further described in PCT Patent Application US2021/041002, previously incorporated herein.

In other examples, the outflow end 1005 of the outer skirt 1002 does not have a folded layer and is releasably secured to an adjacent row of struts in the manner shown in FIGS. 11A-11B (thus, each stitch 262 extends through a single layer of material at the outflow end).

The inflow end 1009 of the skirt 1002 (with or without a fold) can be releasably attached to an adjacent row of struts in the same manner as shown in FIGS. 11A and 11B, using at least one retaining suture 1004, at least one pull suture 1010, and at least one suture forming non-penetrating whip stitches 1008.

In other examples, the outflow end 1005 of the skirt 1002 can have the folded configuration shown in FIGS. 10A, 10B, 11A, and 11B, but is sutured to a row of angled struts without a releasable suture attachment as previously described. For example, the folded outflow end 1005 can be sutured to struts of the frame with a series of whip stitches that extend through layers 1002a, 1002b and around an adjacent strut. Alternatively, the folded outflow end 1005 can be sutured to struts of the frame with a plurality of discrete suture loops that extend through layers 1002a, 1002b and around an adjacent strut. Similarly, the inflow end 1009 of the skirt 1002 (with or without a fold, can be sutured to an adjacent row of angled struts (typically, the row at the inflow end) with a series of ship stitches or discrete suture loops.

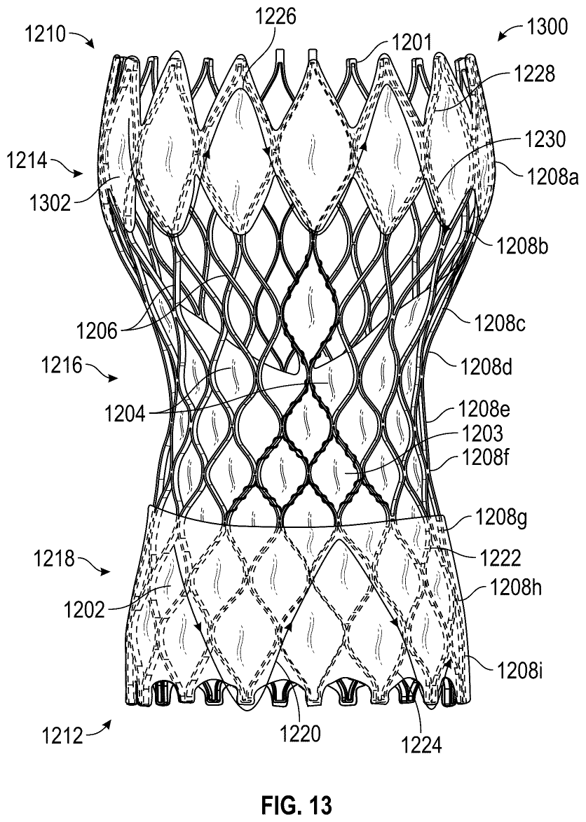

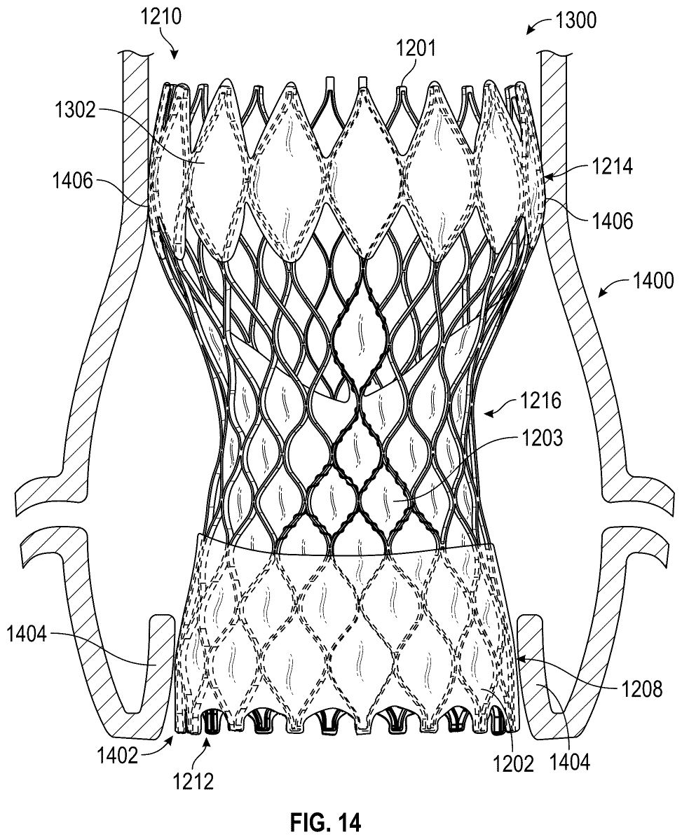

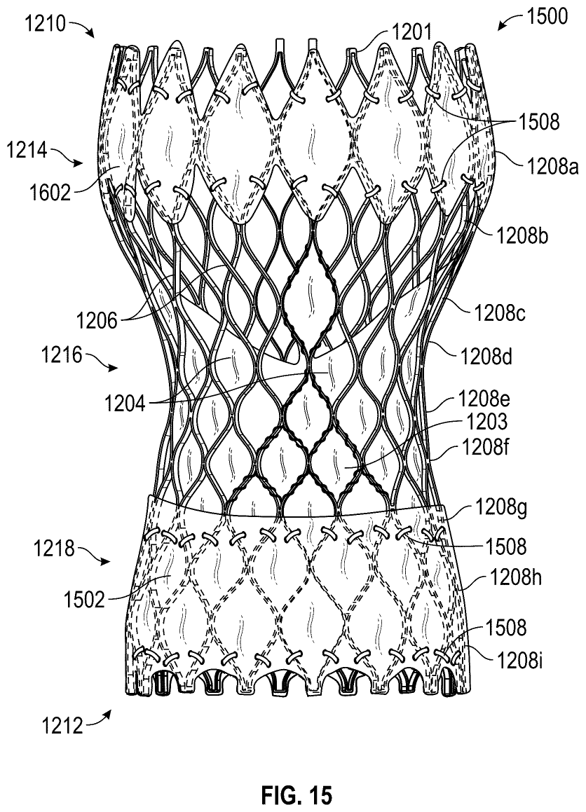

Turning to FIGS. 12-15, additional exemplary prosthetic valves having one or more releasably attached outer skirts are shown and described. As can be seen in FIG. 12, a prosthetic valve 1200 can include a frame 1201, leaflets 1204, an inner skirt 1203, and an outer skirt secured to one end region the frame 1201. As can be seen in FIGS. 13-14, a prosthetic valve 1300 can include the frame 1201, the leaflets 1204, an inner skirt 1203, and the outer skirt 1202 secured to one end region of the frame 1201, and can additionally include an outer skirt 1302 secured to an opposing end region of the frame 1201 relative to the outer skirt 1202. As can be seen in FIG. 15, a prosthetic valve 1500 can include the frame 1201, the leaflets 1204, an inner skirt (not shown), and an outer skirt 1502 secured to one end region of the frame 1201, and an outer skirt 1602 secured to an opposing end region of the frame 1201 relative to the outer skirt 1502.

The frame 1201 can have features similar to other frames described herein (such as, for example, the frames 12, 202, 904, and/or 906). The frame 1201 can comprise a plurality of interconnected struts 1206 which form multiple rows of open cells 1208 between an outflow end 1210 and an inflow end 1212 of the frame 1201. In some examples, a row of cells 1208a is formed by the interconnected struts 1206 at the outflow end 1210 of the frame 1201 and a row of cells 1208i is formed by the interconnected struts 1206 at the inflow end 1212 of the frame 1201. Additional rows of open cells be formed in the frame 1201 between the row of cells 1208a and the row of cells 1208i. For example, as illustrated in FIGS. 12-15, the frame 1201 can include nine rows of cells (rows of cells 1208a-1208i) formed by the interconnected struts 1206.

As shown, the frame 1201 can have an overall hourglass configuration including a flared or bulging outflow end portion 1214, a narrowed central portion 1216, and a flared or bulging inflow end portion 1218. In some examples, the outflow end portion 1214 can include rows of cells 1208a-1208c, the central portion 1216 can include rows of cells 1208d-1208f, and the inflow end portion 1218 can include rows of cells 1208g-1208i. As illustrated in FIGS. 12-15, in some examples, the frame 1201 can have a greatest diameter at the outflow end portion 1214, a smallest diameter at the central portion 1216, and an intermediate diameter (that is, a diameter which is less than the greatest diameter and greater than the smallest diameter) at the inflow end portion 1218. In some examples, the frame can have other proportions for the hourglass shape, for example, having a greatest diameter at the inflow end portion. In some example, each portion can include more or fewer rows of cells.

The hourglass configuration of the frame 1201 can enable each of the outflow end portion 1214 and the inflow end portion 1218 to contact native tissue when implanted in a native heart valve. For example, FIG. 14 shows an illustration of the prosthetic valve 1300 implanted within a native aortic heart valve 1400. As can be seen therein, the inflow end portion 1218 can be seated within an annulus 1402 of the native aortic heart valve 1400 and can contact the native leaflets 1404. Further, the outflow end portion 1214 can be disposed above the annulus 1402 and contact the aortic wall 1406 in the ascending aorta above the coronary arteries. Accordingly, in some examples, the inflow end portion 1218 can be referred to as an annulus portion of the prosthetic valves 1200, 1300, 1500 and the outflow end portion 1214 can be referred to as an aortic portion of the prosthetic valves 1200, 1300, 1500.

The frame 1201 can be made of any of various suitable plastically-expandable materials (for example, stainless steel, etc.) or self-expanding materials (for example, nickel titanium alloy (NiTi), such as nitinol), as known in the art.

In order to facilitate explant of the frame 1201, the frame can include a detachable outer skirts at one or more regions of the frame that contact the native tissue. For example, the prosthetic valve 1200 (FIG. 12) includes the outer skirt 1202 releasably attached to the inflow end portion 1218 of the frame 1201. In another example, the prosthetic valve 1300 (FIGS. 12-14) includes the outer skirt 1202 releasably attached to the inflow end portion 1218 of the frame 1201 and the outer skirt 1302 releasably attached to the outflow end portion 1214 of the frame 1201. In yet another example, the prosthetic valve 1500 (FIG. 15) includes the outer skirt 1502 releasably attached to the inflow end portion 1218 of the frame 1201 and the outer skirt 1602 releasably attached to the outflow end portion 1214 of the frame 1201.

The outer skirts 1202, 1302, 1502, and/or 1602 can include features similar to the outer skirts 18, 402, 502, and 1002 disclosed herein. In some examples, one or more of the outer skirts 1202, 1302, 1502, and/or 1602 can extend around an outer surface of the frame 1201 and can comprise a single sheet of material. In some examples, one or more of the outer skirts 1202, 1302, 1502, and/or 1602 can include multiple overlapping sections (similar to the outer skirt 502) that can each extend over a portion of the outer surface of the frame 1201. In some examples, the outer skirts can be made from a biocompatible fabric, such as a polyethylene terephthalate (PET) fabric. The fabric can be, for example, a woven, knitted, braided fabric, and/or a non-woven fabric (for example, felt). In some examples, one or more of the outer skirts 1202, 1302, 1502, and/or 1602 can be made from a non-fabric material, such as a sheet of material made of a polymer (for example, polyurethane), which can be formed, for example, by extruding, molding or otherwise forming a sheet of material from a liquified polymer. In some examples, one or more of the outer skirts 1202, 1302, 1502, and/or 1602 can be made from natural tissue, such as pericardial tissue (for example, bovine pericardium, porcine pericardium, or pericardium from other sources). The inner skirt 1203 can be made of any of the materials listed above.

In some examples, one or both of the outer skirts 1202 and 1302 can be releasably attached to the frame 1201 via one or more retaining sutures sutured to or looped around selected struts of the frame and one or more pull sutures disposed between the retaining sutures and the exterior surface of the outer skirt, as previously described herein. For example, the outer skirt 1202 can be attached to selected ones of the struts 1206 that form the rows of cells 1208g-1208i. In some examples, the outer skirt 1202 can be attached to the selected struts 1206 via a releasable suture line (including, for example, at least one retaining suture and one or more pull sutures) wherein the suture line (indicated by line 1220) extends along struts of cells 1208i, 1208h, 1208g in a zig-zag or undulating pattern from the inflow end of the outer skirt 1202 to the opposing, outflow end of the outer skirt 1202.

In some examples, the outer skirt 1202 can be attached to selected ones of the struts 1206 that form the rows of cells 1208g-1208i via a first releasable suture line and a second releasable suture line (each including, for example, at least one retaining suture and one or more pull sutures). For example, the first suture line can form a zig-zag or undulating pattern that tracks a row 1222 of struts 1206 forming the upper portion of the row of cells 1208g proximate the outflow edge of the skirt, and the second suture line can form a zig-zag or undulating pattern that tracks a row 1224 of struts 1206 forming the lower portion of the row of cells 1208i and the inflow end 1212 of the frame 1201 proximate the inflow edge of the skirt. In some examples, one or more suture lines can have other configurations or extend over other ones of the struts 1206 to releasably attach the outer skirt 1202 to the frame 1201.

In some examples, the outer skirt 1302 can be releasably attached to the frame 1201 via one or more retaining sutures sutured to or looped around selected struts of the frame and one or more pull suture, as previously described herein. In some examples, the outer skirt 1302 can be attached to selected ones of the struts 1206 that form the rows of cells 1208a-1208b. In some examples, the outer skirt 1302 can be attached to selected struts 1206 that form the rows of cells 1208a-1208b via a releasable suture line (including, for example, at least one retaining suture and one or more pull sutures) wherein the suture line (indicated by line 1226) extends along struts of cells 1208a, 1028b in a zig-zag or undulating pattern from the outflow end of the outer skirt 1202 to the opposing end of the outer skirt 1302.

In some examples, the outer skirt 1302 can be attached to selected one of the struts 1206 that form the rows of cells 1208a-1208b via a first releasable suture line and a second releasable suture line (each including, for example, at least one retaining suture and one or more pull sutures). For example, the first suture line can form a zig-zag or undulating pattern that tracks a row 1228 of struts 1206 forming the upper portion of the row of cells 1208a and the outflow end 1210 of the frame 1201 proximate the outflow edge of the skirt 1302, and the second suture line can form a zig-zag or undulating pattern that tracks a row 1230 of struts forming the lower portion of the row of cell 1208a proximate the inflow edge of the skirt 1302. In some examples, one or more suture lines can have other configurations or extend over other ones of the struts 1206 to releasably attach the outer skirt 1302 to the frame 1201.

As shown in FIG. 13, the upper and lower (outflow and inflow) edges of the outer skirt 1302 can have an undulating shape (for example, zig-zag or saw-tooth shape forming a series of triangular projections as shown) that corresponds of the shape of adjacent rows of angled struts of the frame. In some examples, the upper and lower edges of the outer skirt 1302 can be straight (non-undulating).

In some examples, the outer skirt 1202 can have an undulating shape at a lower (inflow) edge that is proximate to the inflow end 1212 of the frame and a straight upper (outflow) edge as shown. In some examples, the upper edge of the skirt 1202 can have an undulating pattern that corresponds to the shape of the row 1222 of the struts. A height of the skirts 1202 and/or 1302 can vary and/or can cover different portions of the frame than are shown in the illustrated examples of FIGS. 12-14. For example, the skirt 1302 can extend over and cover the row of cells 1208b and optionally the row of cells 1208c (in addition to the row of cells 1208a). In some examples, the upper edge of skirt 1302 can be spaced axially or offset from the outflow end 1210 of the frame toward the inflow end 1212 of the frame.

In some examples, similar to the skirt 1002 shown in FIG. 10A, one or both of the outer skirts 1202, 1302 can include one or more fused or molten edges (such as, for example, the upper edge of the outer skirt 1302, the lower edge of the outer skirt 1302, the upper edge of the outer skirt 1202, and/or the lower edge of the outer skirt 1202. The fused or molten edges may be a region where fibers of the outer skirt are melted and/or fused together to prevent the fibers of the outer skirt material from unraveling and to maintain the woven configuration of the fibers. The fused edges can be oriented away from the leaflets toward the exterior of the prosthetic valve and can be folded over on the exterior surface of the valve to prevent or limit contact between the fused edges and the leaflets of the prosthetic valve, as previously described herein. In some examples, the outer skirts can be releasably sutured to the frame along the fold to secure the position of the fused edges and to enable release of the outer skirts from the frame during, for example, explant of the prosthetic valve, as previously described herein. It will be appreciated the outer skirt including the fused edges can be made from a biocompatible fabric, such as a polyethylene terephthalate (PET) fabric. The fabric can be, for example, a woven, knitted, and/or braided fabric.

In some examples, similar to the illustrations in FIGS. 6A and 6B, the suture line configured for releasable attachment of one or more of the outer skirts 1202 or 1302 can include retaining sutures formed as looped or whip stitches that extend through holes or apertures in the material of the outer skirt, over a pull suture, and around an interior surface of the struts, thereby sandwiching or capturing the material of the outer skirt between the pull suture and an exterior surface of the struts. In some examples, similar to the illustrations of FIGS. 11A and 11B, the suture line can additionally include at least one suture forming a series of non-penetrating whip stitches, where the non-penetrating whip stitches can be secured directly to the strut and the retaining sutures and can be indirectly secured to the strut via the non-penetrating whip stitches. In some examples, the stitches of the retaining suture and/or the whip stitches (if included) can also extend through the inner skirt and/or the cusp edge portions of the leaflets to secure those components to the frame. In some examples, one or both free ends of the pull suture can extend beyond an edge of the outer skirt and/or the frame, and can be attached to the frame and/or the outer skirt, such as attachment via an adhesive, a stitch, tying or knotting the free end to an adjacent strut, an elastic band, and/or other releasable retaining mechanism.

When the pull suture is removed (via for example, pulling on one of the loose ends in a direction parallel to the suture line or outwardly from the frame), the retaining sutures are no longer retained by the pull suture. Thus, as the outer skirts 1202 and/or 1302 are pulled away from the frame 1201 (or the frame is pulled away from the outer skirt), the stitches can be withdrawn through the openings of the respective outer skirt, resulting in the interior face of the outer skirt separating from the strut and the stitches remaining attached to the frame.

In some examples, the retaining sutures can be thinner fibers relative to a thicker pull suture(s). In some examples, the retaining sutures can be comprised of a thinner, high tensile strength biocompatible material, such as an ultra-high molecular weight polyethylene (UHMPE) force fiber® or other similar material or combinations thereof. In one specific example, the retaining sutures can be formed from UHMPE force fiber and can be sufficiently thin and resistant to force such that the retaining sutures can cut through overgrown tissue on the outer surface of the outer skirt as the frame is separated from the outer skirt. In some examples, the pull suture can be comprised of a thicker, high tensile strength, biocompatible material, such as a monofilament comprised of polypropylene (for example, Prolene 4-0), polytetrafluoroethylene (PTFE), expanded PTFE (ePTFE), or other similar materials or combinations thereof. In one specific example, the pull suture can be a microfilament comprised of Prolene, which is configured to readily separate from or be pulled away from any surrounding ingrown tissue when pulled on by the surgeon during an explant procedure.

In some examples, the pull suture material can have a distinct coloration or a colored coating (for example, a green, black, or blue coloration or coating) and/or can be otherwise tagged such that it can be identified by a surgeon during an explant procedure. In some examples, an entire length of the pull suture can, for example, be comprised of a brightly colored material or have a brightly colored coating. In other examples, a portion of the pull suture (for example, a loose end portion of the pull suture) can be comprised of a colored material, have a brightly colored coating, and/or can include a brightly colored tag or flap attached thereto. In other examples, the pull suture or a portion thereof can include a material, coating, or tag that can be visualized using a specialized visualization apparatus. For example, the pull suture can comprise a radiopaque substance or one or more radiopaque markers that can be visualized via a fluoroscopy or x-ray device. For example, the one or more radiopaque markers can be embedded within or attached to an outer surface of the pull suture.

In some examples, the interior face of one or more of the outer skirts 1202 or 1302 may be comprised of a thromboresistant material or surface that can resist tissue ingrowth and improve ease of separation of the outer skirt from the frame, such as those materials discussed above with reference to the outer skirt 502. Further, in some examples, the exterior face of one or more of the outer skirts 1202 or 1302 may be formed in a manner or comprised of a material that encourages tissue ingrowth or may be comprised of the thromboresistant material, such as those materials discussed above with reference to the outer skirt 502. In some examples, the interior surface and/or the exterior surface of one or more of the outer skirts 1202 or 1302 can be coated in a thromboresistant material, such as a hydrogel coating, a heparin coating, a silicone coating, or other materials or combinations thereof, such as those materials discussed above with reference to the outer skirt 502, or can have a smooth laminate liner, such as a polyurethane or polytetrafluoroethylene liner.

It will be appreciated that tissue ingrowth on the exterior face of the outer skirt may enable stable positioning of the prosthetic heart valve while implanted and operating in the native heart valve. It will be further appreciated that use of a thromboresistant material or layer on the interior face of the outer skirt may provide improved separation of the outer skirt from the implantation site and use of a thromboresistant material or layer on the exterior face of the outer skirt may reduce cutting of surrounding tissue during explant.

Returning to the prosthetic valve 1500 shown in FIG. 15, rather than being releasably attached via retaining sutures and pull sutures as in the prosthetic valves 1200, 1300, the outer skirts 1502 and 1602 can be releasably attached to the frame 1201 via bioresorbable couplers 1508. The bioresorbable couplers can comprise a bioresorbable material configured to degrade via bioresorption after implantation of the prosthetic valve 1500. For example, the bioresorbable couplers can comprise poly (L-lactide) (PPLA), polyglycolic acid (PGA), polymer polycaprolactone (PCL), or other bioresorbable materials, or combinations of bioresorbable materials.

In the illustrated example, the bioresorbable couplers 1508 can be sutures that extend over an exterior surface of the outer skirt, through apertures in the outer skirt material (through pre-formed apertures or apertures formed by insertion of the coupler therethrough), and around one of the struts 1206. Further, as illustrated in FIG. 15, the sutures can comprise a first row of sutures along one end region of the outer skirt 1502, 1602 and a second row of sutures along an opposing end region of the outer skirt 1502, 1602, such that the sutures 1508 form two axially spaced suture lines. In the illustrated example, the skirts 1502, 1602 are secured to struts of the frame with sutures 1508 forming discrete, spaced apart stitches. In some examples, the bioresorbable sutures can have other configurations, such as configurations similar to the sutures shown in FIGS. 1 and 4 for attachment of an outer skirt to a frame, or combinations or variations thereof. For example, the bioresorbable sutures can form a plurality of continuous stitches, such as a plurality of whip stitches that extend around one or more struts and through the skirt, similar to sutures 252 or 408. In some examples, the bioresorbable couplers 1508 may comprise other forms of couplers, such as, for example, tabs, hooks, pins, adhesive bodies, or other forms or combinations thereof.

In some examples, the outer skirts 1502, 1602 may be coupled directly to the frame 1201 via the one or more bioresorbable couplers 1508. In some examples, the one or more bioresorbable couplers 1508 may couple the outer skirts 1502, 1602 to the inner skirt (not shown) or to the leaflets 1204 (for example, at an inflow scallop line of the leaflets), or may couple the outer skirts 1502, 1602 to a combination of the frame 1201, the inner skirt, and/or the leaflets 1204. In some examples, other locations of coupling, or combinations of locations of coupling may be utilized.

In some examples, the outer skirts 1502, 1602 may be configured to allow tissue ingrowth with the outer skirt material after implant of the prosthetic valve 1500. The bioresorbable couplers 1508 may be configured to dissolve while positioned within the patient's body, thus reducing the coupling between the outer skirts 1502, 1602 and the frame 1201 (and/or the outer skirts 1502, 1602 and the inner skirt or leaflets) over time. If the outer skirts 1502, 1602 have adhered to the implantation site via tissue adhesion (ingrowth), then the outer skirts 1502, 1602 may be separated more easily during a removal procedure after the bioresorbable couplers 1508 have dissolved. As such, during a removal or explant procedure of the prosthetic valve 1500, the outer skirts 1502, 1602 may be separated from the frame 1201 (and/or the inner skirt or leaflets), to allow such components to be removed from the implantation site while the outer skirts 1502, 1602 remain in place. Thus, the bioresorbable couplers 1508 may enhance the ease of the removal or explant procedure of the prosthetic valve 1500, and/or may reduce the need for cutting of native tissue during a removal or explant procedure of the prosthetic valve 1500.

In some examples, a prosthetic valve can include outer skirts realeasbly attached via a combination of the realeasable attachment mechanisms disclosed herein. For example, an outer skirt can be releasably attached to a frame of a prosthetic valve with one or more bioresorbable couplers and a retaining and pull-suture mechanism (comprising at least one retaining suture and one or more pull sutures). In another example, a first outer skirt can be releasably attached to a portion of a frame (for example, an aortic portion or an annulus portion) of a prosthetic valve with one or more bioresorbable couplers and a second outer skirt can be releasably attached to another portion of the frame (for example, the other of the aortic portion and the annulus portion) via a retaining suture-pull suture mechanism (comprising at least one retaining suture and one or more pull sutures). In some examples, one of the skirts can be releasably attached to a portion of the frame (for example, an aortic portion or an annulus portion) with one or more bioresorbable couplers and/or a retaining and pull-suture mechanism (comprising at least one retaining suture and one or more pull sutures), while the other of the skirts can be attached to the frame using conventional stitching.

Additional details about valve frames, valve structures, inner skirts, outer skirts, and the manner in which a valve structure and/or an outer skirt can be secured to a valve frame via bioresorbable couplers can be found in PCT International Publication No. WO 2022/192500, which is incorporated by reference herein.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (e.g., by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### Additional Examples of the Disclosed Technology

In view of the above-described examples of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic heart valve comprising: an annular frame comprising a plurality of interconnected struts; and an outer skirt disposed on an outer surface of the annular frame and detachably secured to at least a portion of the plurality of interconnected struts. The outer skirt is detachably secured to the portion of the plurality of interconnected struts via at least one retaining suture and one or more pull sutures, the retaining suture and one or more pull sutures arranged along a suture line, the retaining suture forming a plurality of stitches extending around the portion of the plurality of interconnected struts, through the outer skirt, and over the one or more pull sutures. The outer skirt is configured to detach from the portion of the plurality of interconnected struts and enable separation of the annular frame from the outer skirt via removal of the one or more pull sutures from the suture line and withdrawal of the stitches through the outer skirt.

Example 2. The prosthetic heart valve of any example herein, particularly example 1, wherein each of the one or more pull sutures comprises an end region that extends beyond the suture line, the end region being attached to an attachment region at one or more of the annular frame, the outer skirt, or a valvular structure disposed within an interior of the annular frame; and wherein the end region is configured to be released from the attachment region and pulled to remove a corresponding pull suture of the one or more pull sutures from the suture line.

Example 3. The prosthetic heart valve of any example herein, particularly example 2, wherein the end region is releasably attached at the attachment region via one or more of an adhesive, a suture, a knot, or an elastic band.

Example 4. The prosthetic heart valve of any example herein, particularly either of examples 2 or 3, wherein the end region comprises a marker configured to enable identification of a corresponding pull suture of the one or more pull sutures.

Example 5. The prosthetic heart valve of any example herein, particularly example 4, wherein, the marker comprises at least one of a colored marker or a radiopaque marker attached to or disposed within the end region.

Example 6. The prosthetic heart valve of any example herein, particularly anyone of examples 1-5, wherein the outer skirt comprises a first skirt portion and a second skirt portion, the first skirt portion detachably secured to a first portion of the plurality of interconnected struts via at least a first pull suture and at least one first retaining suture forming a first set of stitches, the second skirt portion detachably secured to a second portion of the plurality of interconnected struts via at least a second pull suture and at least one second retaining suture forming a second set of stitches; and wherein the prosthetic heart valve is configured to enable separation of a first portion of the annular frame from the first skirt portion via at least removal of at least the first pull suture from the suture line and withdrawal of the first set of stitches through the outer skirt, and the prosthetic heart valve is further configured to enable separation of a second portion of the annular frame from the second skirt portion via removal of at least the second pull suture and withdrawal of the second set of stitches through the outer skirt.

Example 7. The prosthetic heart valve of any example herein, particularly example 6, wherein the outer skirt further comprises a third skirt portion detachably secured to a third portion of the plurality of interconnected struts via at least a third pull suture and at least one third retaining suture forming a third set of stitches; and wherein the prosthetic heart valve is configured to enable separation of a third portion of the annular frame from the third skirt portion via removal of at least the third pull suture from the suture line and withdrawal of the third set of stitches through the outer skirt.

Example 8. The prosthetic heart valve of any example herein, particularly example 7, further comprising a valvular structure comprising a plurality of leaflets disposed within an interior of the annular frame, wherein the plurality of leaflets comprise a first leaflet, as second leaflet, and a third leaflet; and wherein the first skirt portion is detachably secured to the first portion of the plurality of interconnected struts in a position aligned with the first leaflet, the second skirt portion is detachably secured to the second portion of the plurality of interconnected struts in a position aligned with the second leaflet, and the third skirt portion is detachably secured to the third portion of the plurality of interconnected struts in a position aligned with the third leaflet.

Example 9. The prosthetic heart valve of any example herein, particularly either of examples 7 or 8, wherein the prosthetic heart valve is configured for step-wise separation of the first, second, and third portions of the annular frame the outer skirt during an explant procedure of the prosthetic heart valve.

Example 10. The prosthetic heart valve of any example herein, particularly any one of examples 1-9, wherein the outer skirt comprises a material configured to enable tissue ingrowth therein.

Example 11. The prosthetic heart valve of any example herein, particularly any one of examples 1-9, wherein the outer skirt comprises a material configured to discourage tissue ingrowth therein.

Example 12. The prosthetic heart valve of any example herein, particularly any one of examples 1-11, wherein the one or more pull sutures are comprised of one or more of PTFE, ePTFE, or prolene.

Example 13. The prosthetic heart valve of any example herein, particularly any one of examples 1-12, wherein the one or more pull sutures comprise an identifying material or coating, and wherein the identifying material or coating is configured to enable identification of the one or more pull sutures during an explant procedure of the prosthetic heart valve, the identification material or coating comprising at least one of a colored material or coating or a radiopaque material or coating.

Example 14. The prosthetic heart valve of any example herein, particularly any one of examples 1-13, wherein the at least one retaining suture comprises UHMPE force fiber.

Example 15. The prosthetic heart valve of any example herein, particularly any one of examples 1-14, wherein the outer skirt is disposed on the outer surface of the annular frame around a circumference thereof.

Example 16. The prosthetic heart valve of any example herein, particularly any one of examples 1-15, wherein the at least one retaining suture does not extend through the one or more pull sutures.

Example 17. The prosthetic heart valve of any example herein, particularly any one of examples 1-16, wherein the at least one retaining suture extends through preformed holes in the outer skirt.

Example 18. The prosthetic heart valve of any example herein, particularly any one of examples 1-17, wherein each stitch extends through a single opening in the outer skirt.

Example 19. The prosthetic heart valve of any example herein, particularly any one of examples 1-18, wherein the stitches are configured to remain in place attached to the portion of the plurality of interconnected struts after separation of the annular frame from the outer skirt during an explant procedure.

Example 20. The prosthetic heart valve of any example herein, particularly any one of examples 1-19, wherein the outer skirt comprises two or more skirt portions, adjacent ones of the two or more skirt portions having an overlapping region therebetween, the suture line extended along the overlapping region.

Example 21. A prosthetic heart valve comprising: a frame comprising a plurality of interconnected struts arranged between an inflow end and an outflow end of the frame; an outer skirt disposed on an exterior surface of the frame; one or more pull sutures disposed on an exterior surface of the outer skirt along a suture line; and at least one retaining suture forming a plurality of stitches looped around the one or more pull sutures and specified ones of the plurality of interconnected struts disposed along the suture line, wherein material of the outer skirt is captured between the pull suture and the exterior surface of the frame along the suture line. The outer skirt is releaseably attached to the frame via the at least one retaining suture and the one or more pull sutures. The outer skirt is configured to be released from the frame via removal of the one or more pull sutures from the plurality of stitches along the suture line, and withdrawal of the stitches through the outer skirt along the suture line. The plurality of stitches are configured to remain looped around the specified ones of the plurality of interconnected struts along the suture line after the release of the outer skirt from the frame.

Example 22. The prosthetic heart valve of any example herein, particularly example 21, wherein each of the one or more pull sutures comprises an end that extends beyond the suture line, the end attached to an attachment region at one or more of the frame, the outer skirt, or a valvular structure disposed within an interior of the frame; and wherein the end is configured to be released from the attachment region and pulled to remove a corresponding pull suture of the one or more pull sutures from the suture line.

Example 23. The prosthetic heart valve of any example herein, particularly example 22, wherein the end is temporarily attached at the attachment region via one or more of an adhesive, a suture, a knot, or an elastic band.

Example 24. The prosthetic heart valve of any example herein, particularly any one of examples 21-23, wherein at least a portion of each of the one or more pull sutures is comprised of an identifying material or coating, and wherein the identifying material or coating is configured to enable identification of the one or more pull sutures during an explant procedure of the prosthetic heart valve, the identification material or coating comprising at least one of a colored material or coating or a radiopaque material or coating

Example 25. The prosthetic heart valve of any example herein, particularly any one of examples 21-24, wherein the outer skirt comprises two or more skirt portions, adjacent ones of the two or more skirt portions having an overlapping region therebetween, the suture line extended along the overlapping region.

Example 26. The prosthetic heart valve of any example herein, particularly any one of examples 21-25, wherein the outer skirt comprises a first skirt portion and a second skirt portion, the first skirt portion detachably secured to a first portion of the plurality of interconnected struts via at least a first pull suture and at least one first retaining suture forming a first set of stitches, the second skirt portion detachably secured to a second portion of the plurality of interconnected struts via at least a second pull suture and at least one second retaining suture forming a second set of stitches; and wherein the prosthetic heart valve is configured to enable separation of a first portion of the frame from the first skirt portion via at least removal of at least the first pull suture from the suture line and withdrawal of the first set of stitches through the outer skirt, and the prosthetic heart valve is further configured to enable separation of a second portion of the frame from the second skirt portion via removal of at least the second pull suture and withdrawal of the second set of stitches through the outer skirt

Example 27. The prosthetic heart valve of any example herein, particularly example 26, wherein the outer skirt further comprises a third skirt portion detachably secured to a third portion of the plurality of interconnected struts via at least a third pull suture and at least one third retaining suture forming a third set of stitches; and wherein the prosthetic heart valve is configured to enable separation of a third portion of the frame from the third skirt portion via removal of at least the third pull suture from the suture line and withdrawal of the third set of stitches through the outer skirt.

Example 28. The prosthetic heart valve of any example herein, particularly any one of examples 21-27, wherein an interior surface of the outer skirt comprises a thromboresistant material, and wherein an exterior surface of the outer skirt comprises a material configured to encourage tissue ingrowth therein.

Example 29. The prosthetic heart valve of any example herein, particularly any one of examples 21-28, wherein the at least one retaining suture is thinner than the one or more pull sutures.

Example 30. A method of explanting a prosthetic heart valve, the method including a plurality of steps comprising: accessing an implantation site of the prosthetic heart valve, the prosthetic heart valve comprising a frame having an outer skirt detachably secured on an exterior surface thereof, the outer skirt detachably secured to the annular frame via at least one pull suture and at least one retaining suture forming a plurality of stitches looped around the pull suture and further looped around one or more struts of the frame; grasping an end of the pull suture, and applying a force to the end to withdraw the pull suture from the stitches; separating the frame from one or more portions of the outer skirt; and removing the frame from the implantation site.

Example 31. The method of any example herein, particularly example 30, further comprising identifying a marker associated with the pull suture, the marker comprising at least one of a colored material or coating or a radiopaque marker or coating.

Example 32. The method of any example herein, particularly either of examples 30 or 31, further comprising freeing, from an attachment, the end of the pull suture.

Example 33. The method of any example herein, particularly example 32, wherein the freeing the end comprises at least one of cutting or pulling an exposed portion of the end to release the attachment.

Example 34. The method of any example herein, particularly any one of examples 30-33, wherein the separating the frame from the one or more portions of the outer skirt comprises, after the withdrawal of the pull suture, applying a force on the frame to withdraw the plurality of retaining sutures through material of the outer skirt.

Example 35. The method of any example herein, particularly example 34, wherein the applying the force on the frame to withdraw the stitches through the outer skirt results in one or more of the stitches cutting through overgrown tissue at an exterior surface of one or more of the outer skirt.

Example 36. The method of any example herein, particularly any one of examples 30-35, wherein the outer skirt comprises a first portion, a second portion, and a third portion releasably attached to the frame via a first pull suture, a second pull suture, and a third pull suture, respectively, each having a portion of the stitches looped therearound, and wherein the applying a force to the loose end to withdraw the pull suture and the separating the frame from the one or more portions of the outer skirt comprises step-wise withdrawal of the first pull suture and separation of at least a section of the first portion of the outer skirt, withdrawal of the second pull suture and separation of at least a section of the second portion of the outer skirt, and withdrawal of the third pull suture and separation of at least a section of the third portion of the outer skirt.

Example 37. The method of any example herein, particularly any one of examples 30-36, further comprising, prior to the removing of the frame from the implantation site, radially compressing or folding the frame to reduce a diameter of the frame.

Example 38. The method of any example herein, particularly any one of examples 30-37, further comprising, prior to the removing of the outer skirt from the implantation site, cutting one or more of tissue ingrown into the outer skirt or a native leaflet at the implantation site.

Example 39. The method of any example herein, particularly any one of examples 30-38, further comprising, after removal of the frame, removing the one or more portions of the outer skirt from the implantation site.

Example 40. The method of any example herein, particularly any one of examples 30-39, wherein the plurality of steps are performed in an open-heart surgical procedure.

Example 41. The method of any example herein, particularly any one of examples 30-39, wherein the plurality of steps are performed in a transcatheter procedure.

Example 42. A method of manufacturing a prosthetic heart valve having a detachable outer skirt, the method comprising: obtaining a frame comprising a plurality of interconnected struts arranged between an inflow end and an outflow end of the frame; overlaying one or more portions of the detachable outer skirt on an exterior surface of the frame; and a plurality of stitches around selected ones of the plurality of interconnected struts, through the detachable outer skirt, and over one or more pull sutures on an exterior surface of the detachable outer skirt.

Example 43. The method of any example herein, particularly example 42, further comprising, for each of the one or more pull sutures, forming an attachment between an end of a pull suture and an attachment region on one of the detachable outer skirt, the frame, or a valvular structure disposed within an interior of the frame.

Example 44. The method of any example herein, particularly example 43, wherein the forming the attachment comprises capturing the end within the attachment region via at least one of an adhesive, a suture, a knot, or an elastic material.

Example 45. The method of any example herein, particularly any one of examples 42-44, wherein the overlaying the one or more portions of the detachable outer skirt on the exterior surface of the frame comprises respectively aligning each of a first portion, a second portion, and a third portion of the detachable outer skirt with a cusp edge of a first leaflet, a cusp edge of a second leaflet, and a cusp edge a third leaflet, respectively, of a valvular structure disposed within an interior of the frame.

Example 46. The method of any example herein, particularly example 45, wherein the one or more pull sutures comprise a first pull suture configured to enable detachment of at least the first portion of the detachable outer skirt, a second pull suture configured to enable detachment of at least the second portion of the detachable outer skirt, and a third pull suture configured to enable detachment of at least the third portion of the detachable outer skirt.

Example 47. The method of any example herein, particularly any one of examples 42-46, wherein the overlaying the one or more portions of the detachable outer skirt on the exterior surface of the frame comprises overlapping adjacent edges of the one or more portions of the detachable outer skirt for form an overlapping region therebetween.

Example 48. The method of any example herein, particularly example 47, wherein the forming of stitches comprises forming stitches along the overlapping region.

Example 49. The method of any example herein, particularly any one of examples 42-48, wherein the forming of stitches comprises threading a suture through the outer skirt such that each stitch extends through a single opening in the outer skirt.

Example 50. The method of any example herein, particularly example, 49, wherein the stitches comprise whip stitches.

Example 51. The method of any example herein, particularly any one of examples 49-50, wherein the outer skirt comprises a plurality of pre-formed openings that receive the stitches.

Example 52. The method of any example herein, particularly any one of examples 49-50, wherein the openings are formed when forming the stitches.

Example 53. A method of implanting a prosthetic heart valve, the method including a plurality of steps comprising: evaluating a heart condition of a patient and size of a native annulus of the patient; based on the heart condition of the patient and size of the native annulus, selecting a prosthetic heart valve having an appropriate size for implantation within the patient, the prosthetic heart valve comprising a frame having an outer skirt detachably secured on an exterior surface thereof, the outer skirt detachably secured to the frame via at least one pull suture and at least one retaining suture forming a plurality of stitches looped around the pull suture and further looped around one or more struts of the frame; if the heart condition of the patient is aortic insufficiency, removing the outer skirt from the frame by grasping an end of the pull suture, and applying a force to the end to withdraw the pull suture from the stitches, and separating the frame from one or more portions of the outer skirt; and implanting the prosthetic heart valve within the native annulus of the patient.

Example 54. The method of any example herein, particularly example 53, further comprising identifying a marker associated with the pull suture, the marker comprising at least one of a colored material or coating.

Example 55. The method of any example herein, particularly any one of examples 53 or 54, further comprising freeing, from an attachment, the end of the pull suture.

Example 56. The method of any example herein, particularly example 55, wherein the freeing the end comprises at least one of cutting or pulling an exposed portion of the end to release the attachment.

Example 57. The method of any example herein, particularly any one of examples 53-56, wherein the separating the frame from the one or more portions of the outer skirt comprises, after the withdrawal of the pull suture, applying a force on at least one of the outer skirt or the frame to withdraw the stitches through the outer skirt.

Example 58. The method of any example herein, particularly any one of examples 53-57, wherein the outer skirt comprises a first portion, a second portion, and a third portion releasably attached to the frame via a first pull suture, a second pull suture, and a third pull suture, respectively, each having a portion of the stitches looped therearound, and wherein the applying a force to the loose end to withdraw the pull suture and the separating the frame from the one or more portions of the outer skirt comprises step-wise withdrawal of the first pull suture and separation of at least a section of the first portion of the outer skirt, withdrawal of the second pull suture and separation of at least a section of the second portion of the outer skirt, and withdrawal of the third pull suture and separation of at least a section of the third portion of the outer skirt.

Example 59. The method of any example herein, particularly any one of examples 53-58, wherein the implanting the prosthetic heart valve comprises implanting the prosthetic heart valve in a transcatheter procedure.

Example 60. A prosthetic heart valve comprising: an annular frame comprising a plurality of interconnected struts and a plurality of anchoring projections; an outer skirt disposed on an outer surface of the annular frame and covering at least a portion of the plurality of projections, the outer skirt detachably secured to at least a portion of the plurality of interconnected struts; wherein the outer skirt is detachably secured to the portion of the plurality of interconnected struts via at least one retaining suture and one or more pull sutures, the retaining suture and one or more pull sutures arranged along a suture line, the retaining suture forming a plurality of stitches extending around the portion of the plurality of interconnected struts, through the outer skirt, and over the one or more pull sutures; and wherein the outer skirt is configured to detach from the portion of the plurality of interconnected struts and enable separation of the annular frame from the outer skirt via removal of the one or more pull sutures from the suture line and withdrawal of the plurality of stitches through the outer skirt, the plurality of projections on the outer surface of the annular frame being exposed after separation of the frame and the outer skirt.

Example 61. The prosthetic heart valve of any example herein, particularly example 60, wherein each of the one or more pull sutures comprises an end that extends beyond the suture line, the end being attached to an attachment region at one or more of the annular frame, the outer skirt, or a valvular structure disposed within an interior of the annular frame; and wherein the end is configured to be released from the attachment region and pulled to remove a corresponding pull suture of the one or more pull sutures from the suture line.

Example 62. The prosthetic heart valve of any example herein, particularly example 61, wherein the end is temporarily attached at the attachment region via one or more of an adhesive, a suture, a knot, or an elastic band.

Example 63. The prosthetic heart valve of any example herein, particularly any one of examples 61 or 62, wherein the end comprises a marker attached thereto or disposed therein which is configured to enable identification of a corresponding pull suture of the one or more pull sutures.

Example 64. The prosthetic heart valve of any example herein, particularly example 63, wherein the marker comprises a colored marker.

Example 65. The prosthetic heart valve of any example herein, particularly any one of examples 60-64, wherein the outer skirt comprises a first skirt portion and a second skirt portion, the first skirt portion detachably secured to a first portion of the plurality of interconnected struts via at least a first pull suture and at least one first retaining suture forming a first set of stitches, the second skirt portion detachably secured to a second portion of the plurality of interconnected struts via at least a second pull suture and at least one second retaining suture forming a second set of stitches; and wherein the prosthetic heart valve is configured to enable separation of a first portion of the annular frame from the first skirt portion via at least removal of at least the first pull suture from the suture line and withdrawal of the first set of stitches through the outer skirt, and the prosthetic heart valve is further configured to enable separation of a second portion of the annular frame from the second skirt portion via removal of at least the second pull suture and withdrawal of the second set of stitches through the outer skirt.

Example 66. The prosthetic heart valve of any example herein, particularly example 65, wherein the outer skirt further comprises a third skirt portion detachably secured to a third portion of the plurality of interconnected struts via at least a third pull suture and at least one third retaining suture forming a third set of stitches; and wherein the prosthetic heart valve is configured to enable separation of a third portion of the annular frame from the third skirt portion via removal of at least the third pull suture from the suture line and withdrawal of the third set of stitches through the outer skirt.

Example 67. The prosthetic heart valve of any example herein, particularly example 66, further comprising a valvular structure comprising a plurality of leaflets disposed within an interior of the annular frame, wherein the plurality of leaflets comprise a first leaflet, as second leaflet, and a third leaflet; and wherein the first skirt portion is detachably secured to the first portion of the plurality of interconnected struts in a position aligned with the first leaflet, the second skirt portion is detachably secured to the second portion of the plurality of interconnected struts in a position aligned with the second leaflet, and the third skirt portion is detachably secured to the third portion of the plurality of interconnected struts in a position aligned with the third leaflet.

Example 68. The prosthetic heart valve of any example herein, particularly any one of examples 60-67, wherein the outer skirt comprises a material configured to enable tissue ingrowth therein.

Example 69. The prosthetic heart valve of any example herein, particularly any one of examples 60-68, wherein the outer skirt comprises a material configured to fill in one or more gaps within calcified tissue of a native heart valve.

Example 70. The prosthetic heart valve of any example herein, particularly any one of examples 60-69, wherein the one or more pull sutures are comprised of one or more of PTFE, ePTFE, or prolene.

Example71. The prosthetic heart valve of any one of claims 60-70, wherein the one or more pull sutures comprise an identifying material or coating, and wherein the identifying material or coating is configured to enable identification of the one or more pull sutures prior to an implant procedure of the prosthetic heart valve, the identification material or coating comprising a colored material or coating.

Example 72. The prosthetic heart valve of any example herein, particularly any one of examples 60-71, wherein the at least one retaining suture comprises UHMPE force fiber.

Example 73. The prosthetic heart valve of any example herein, particularly any one of examples 60-72, wherein the outer skirt is disposed on the outer surface of the annular frame around a circumference thereof.

Example 74. The prosthetic heart valve of any example herein, particularly any one of examples 60-73, wherein the at least one retaining suture does not extend through the one or more pull sutures.

Example 75. The prosthetic heart valve of any example herein, particularly any one of examples 60-74, wherein the at least one retaining suture extends through preformed openings in the outer skirt.

Example 76. The prosthetic heart valve of any example herein, particularly any one of examples 60-75, wherein each stitch extends through a single opening in the outer skirt.

Example 77. The prosthetic heart valve of any example herein, particularly any one of examples claims 60-76, wherein the stitches are configured to remain in place attached to the portion of the plurality of interconnected struts after separation of the annular frame from the outer skirt.

Example 78. The prosthetic heart valve of any example herein, particularly any one of examples 60-77, wherein the outer skirt comprises two or more skirt portions, adjacent ones of the two or more skirt portions having an overlapping region therebetween, the suture line extended along the overlapping region.

Example 79. The prosthetic heart valve of any example herein, particularly any one of examples 60-78, wherein the outer skirt is configured to be selectively removable for treatment of a patient with aortic insufficiency, and wherein the outer skirt is configured to remain attached to the annular frame for treatment of a patient with aortic stenosis.

Example 80. A method of implanting a prosthetic heart valve, the method including a plurality of steps comprising: evaluating a heart condition of a patient and size of a native annulus of the patient; based on the heart condition of the patient and size of the native annulus, selecting a prosthetic heart valve having an appropriate size for implantation within the patient, the prosthetic heart valve comprising a frame having an outer skirt secured on an exterior surface thereof; if the heart condition of the patient is aortic insufficiency, removing the outer skirt from the frame; and implanting the prosthetic heart valve within the native annulus of the patient.

Example 81. The method of any example herein, particularly example 80, wherein the implanting the prosthetic heart valve causes a plurality of anchoring projections on the frame to engage the native annulus.

Example 82. The method of any example herein, particularly any one of examples 80-81, wherein the outer skirt is detachably secured to the frame via at least one pull suture and at least one retaining suture forming a plurality of stitches looped around the pull suture and further looped around one or more struts of the frame; and wherein removing the outer skirt from the frame comprises: grasping an end of the pull suture; applying a force to the end to withdraw the pull suture from the stitches; and separating the frame from one or more portions of the outer skirt.

Example 83. The method any example herein, particularly example 82, further comprising identifying a marker associated with the pull suture, the marker comprising at least one of a colored material or coating.

Example 84. The method of any example herein, particularly any one of examples 82 or 83, further comprising freeing, from an attachment, the end of the pull suture, wherein the freeing the end comprises at least one of cutting or pulling an exposed portion of the end to release the attachment.

Example 85. The method of any example herein, particularly any one of examples 82-84, wherein the separating the frame from the one or more portions of the outer skirt comprises, after the withdrawal of the pull suture, applying a force on at least one of the outer skirt or the frame to withdraw the stitches through the outer skirt.

Example 86. The method of any example herein, particularly any one of examples 80-85, wherein the implanting the prosthetic heart valve comprises implanting the prosthetic heart valve in a transcatheter procedure.

Example 87. A method of implanting a prosthetic heart valve, the method including a plurality of steps comprising: evaluating a heart condition of a patient and size of a native annulus of the patient; based on the heart condition of the patient and size of the native annulus, selecting a prosthetic heart valve having an appropriate size for implantation within the patient, the prosthetic heart valve comprising a frame having an outer skirt secured on an exterior surface thereof; if the heart condition of the patient is aortic insufficiency, removing the outer skirt from the frame and implanting the prosthetic heart valve within the native annulus of the patient without the outer skirt; and if the heart condition of the patient is aortic stenosis, implanting the prosthetic heart valve within the native annulus of the patient with the outer skirt.

Example 88. The method of any example herein, particularly example 87, wherein the outer skirt is detachably secured to the frame via at least one pull suture and at least one retaining suture forming a plurality of stitches looped around the pull suture and further looped around one or more struts of the frame; and wherein the frame comprises a plurality of anchoring projections covered by the outer skirt.

Example 89. The method of any example herein, particularly example 88, wherein removing the outer skirt from the frame comprises: grasping an end of the pull suture; applying a force to the end to withdraw the pull suture from the stitches; and separating the frame from one or more portions of the outer skirt.

Example 90. The method of any example herein, particularly any one of examples 88 or 89, wherein the implanting the prosthetic heart valve, if the heart condition of the patient is aortic insufficiency, causes the plurality of anchoring projections on the frame to engage the native annulus.

Example 91. The method of any example herein, particularly example 88, wherein the implanting the prosthetic heart valve, if the heart condition of the patient is aortic stenosis, causes the outer skirt to contact the native annulus and limit contact between the native annulus and the plurality of anchoring projections on the frame.

Example 92. A prosthetic heart valve comprising: an annular frame comprising a plurality of interconnected struts and a plurality of anchoring projections; and an outer skirt disposed on an outer surface of the annular frame and covering at least a portion of the plurality of projections, the outer skirt detachably secured to at least a portion of the plurality of interconnected struts; wherein the outer skirt is configured to detach from the portion of the plurality of interconnected struts and enable separation of the annular frame from the outer skirt, the plurality of projections on the outer surface of the annular frame being exposed after separation of the frame and the outer skirt.

Example 93. The prosthetic heart valve of any example herein, particularly example 92, wherein the outer skirt is detachably secured to the portion of the plurality of interconnected struts via at least one retaining suture and one or more pull sutures, the retaining suture and one or more pull sutures arranged along a suture line, the retaining suture forming a plurality of stitches extending around the portion of the plurality of interconnected struts, through the outer skirt, and over the one or more pull sutures, and wherein the outer skirt can be detached from the frame via removal of the one or more pull sutures from the suture line and withdrawal of the plurality of stitches through the outer skirt.

Example 94. The prosthetic heart valve of any example herein, particularly example 93, wherein each of the one or more pull sutures comprises an end region that extends beyond the suture line, the end region being releasably attached to an attachment region at one or more of the annular frame, the outer skirt, or a valvular structure disposed within an interior of the annular frame.

Example 95. The prosthetic heart valve of any example herein, particularly example 94, wherein the end is configured to be released from the attachment region and pulled to remove a corresponding pull suture of the one or more pull sutures from the suture line.

Example 96. The prosthetic heart valve of any example herein, particularly any one of examples 93-95, wherein at least a portion of each of the one or more pull sutures comprise an identifying material or coating, and wherein the identifying material or coating is configured to enable identification of the one or more pull sutures prior to an implant procedure of the prosthetic heart valve, the identification material or coating comprising a colored material or coating.

Example 97. A prosthetic heart valve comprising: an annular frame comprising a plurality of interconnected struts; and an outer skirt disposed on an outer surface of the annular frame, wherein the outer skirt comprises at least one fused edge; wherein the fused edge is folded over an exterior surface of the prosthetic heart valve to create a folded edge, wherein the folded edge is secured to adjacent struts of the plurality of interconnected struts.

Example 98. The prosthetic valve of any example herein, particularly example 97, wherein the outer skirt is detachably secured to the portion of the plurality of interconnected struts along the folded edge via at least one retaining suture and one or more pull sutures, the retaining suture and one or more pull sutures arranged along a suture line, the retaining suture forming a plurality of stitches extending through the outer skirt and over the one or more pull sutures.

Example 99. The prosthetic heart valve of any example herein, particularly example 98, wherein the outer skirt is configured to detach from the portion of the plurality of interconnected struts along the folded edge and enable separation of the annular frame from the outer skirt via removal of the one or more pull sutures from the suture line and withdrawal of the plurality of stitches through the outer skirt.

Example 100. The prosthetic heart valve of any example herein, particularly example 99, wherein each of the one or more pull sutures comprises an end region that extends beyond the suture line, the end region being attached to an attachment region at one or more of the annular frame, the outer skirt, or a valvular structure disposed within an interior of the annular frame; and wherein the end region is configured to be released from the attachment region and pulled to remove a corresponding pull suture of the one or more pull sutures from the suture line.

Example 101. The prosthetic heart valve of any example herein, particularly any of examples 98-100, wherein the at least one retaining suture extends through preformed openings in the outer skirt.

Example 102. The prosthetic heart valve of any example herein, particularly any of examples 98-101, wherein the outer skirt is further detachably secured to the portion of the plurality of interconnected struts along the folded edge via a plurality of non-penetrating stitches, the plurality of non-penetrating stitches extending around the portion of the plurality of interconnected struts and between the at least one retaining suture and an interior surface of the outer skirt.

Example 103. The prosthetic heart valve of any example herein, particularly example 102, wherein the stitches and the non-penetrating stitches are configured to remain in place attached to the portion of the plurality of interconnected struts after separation of the annular frame from the outer skirt during an explant procedure.

Example 104. A prosthetic heart valve comprising: an annular frame comprising a plurality of interconnected struts; and an outer skirt disposed on an outer surface of the annular frame and detachably secured to at least a portion of the plurality of interconnected struts; wherein the outer skirt is detachably secured to the portion of the plurality of interconnected struts via at least one retaining suture, one or more pull sutures, and a plurality of non-penetrating stitches, the retaining suture, the one or more pull sutures, and the plurality of non-penetrating stitches arranged along a suture line, the retaining suture forming a plurality of stitches extending through the outer skirt and over the one or more pull sutures, the non-penetrating stitches extending around the portion of the plurality of interconnected struts and between the at least one retaining suture and an interior surface of the outer skirt; and wherein the outer skirt is configured to detach from the portion of the plurality of interconnected struts and enable separation of the annular frame from the outer skirt via removal of the one or more pull sutures from the suture line and withdrawal of the plurality of stitches through the outer skirt.

Example 105. The prosthetic heart of any example herein, particularly example 104, wherein the stitches and the non-penetrating stitches are configured to remain in place attached to the portion of the plurality of interconnected struts after separation of the annular frame from the outer skirt during an explant procedure.

Example 106. The prosthetic heart valve of any example herein, particularly either of examples 104 or 105, wherein each of the one or more pull sutures comprises an end that extends beyond the suture line, the end being attached to an attachment region at one or more of the annular frame, the outer skirt, or a valvular structure disposed within an interior of the annular frame; and wherein the end is configured to be released from the attachment region and pulled to remove a corresponding pull suture of the one or more pull sutures from the suture line.

Example 107. The prosthetic heart valve of any example herein, particularly any one of examples 104-106, wherein the at least one retaining suture extends through preformed openings in the outer skirt.

Example 108. The prosthetic heart valve of any example herein, particularly any one of examples 104-107, wherein the outer skirt comprises at least one fused edge, and the fused edge is folded over an exterior surface of the prosthetic heart valve to create a folded edge; and wherein the outer skirt is detachably secured to the portion of the plurality of interconnected struts along the folded edge.

Example 108. The prosthetic heart valve of any example herein, particularly any one of examples 104-107, wherein the outer skirt comprises at least one fused edge, and the fused edge is folded over an exterior surface of the prosthetic heart valve to create a folded edge; and wherein the outer skirt is detachably secured to the portion of the plurality of interconnected struts along the folded edge.

Example 109. The prosthetic heart valve of any example herein, particularly example 108, wherein the fused edge is one of the outflow edge or the inflow edge of the outer skirt.

Example 110. The prosthetic heart valve of any example herein, particularly any of examples 104-107, wherein the outflow edge of the outer skirt is detachably secured to the portion of the plurality of interconnected struts.

Example 111. The prosthetic heart valve of any example herein, particularly any of examples 104-107, wherein the inflow edge of the outer skirt is detachably secured to the portion of the plurality of interconnected struts.

Example 112. A prosthetic heart valve comprising: an annular frame comprising a plurality of interconnected struts; a first outer skirt disposed on an outer surface of an inflow end region of the annular frame and detachably secured to at least a first portion of the plurality of interconnected struts; and a second outer skirt disposed on an outer surface of an outflow end region of the annular frame and detachably secured to at least a second portion of the plurality of interconnected struts.

Example 113. The prosthetic heart valve of any example herein, particularly example 112, wherein the inflow end portion is an annulus portion configured to be seated in a native annulus and the outflow end portion is an aortic portion configured to contact an aortic wall when the prosthetic heart valve is implanted in a native heart valve.

Example 114. The prosthetic heart valve of any example herein, particularly either of examples 112 or 113, wherein the first outer skirt is detachably secured to the first portion of the plurality of interconnected struts via at least one retaining suture and one or more pull sutures, the retaining suture and one or more pull sutures arranged along a suture line, the retaining suture forming a plurality of stitches extending around the first portion of the plurality of interconnected struts, through the first outer skirt, and over the one or more pull sutures.

Example 115. The prosthetic heart valve of any example herein, particularly example 114, wherein each of the first outer skirt is configured to detach from the first portion of the plurality of interconnected struts and enable separation of the annular frame from the first outer skirt via removal of the one or more pull sutures from respective the suture lines and withdrawal of the stitches through the first outer skirt.

Example 116. The prosthetic heart valve of any example herein, particularly any of examples 112-115, wherein the second outer skirt is detachably secured to the second portion of the plurality of interconnected struts via at least one retaining suture and one or more pull sutures, the retaining suture and one or more pull sutures arranged along a suture line, the retaining suture forming a plurality of stitches extending around the second portion of the plurality of interconnected struts, through the second outer skirt, and over the one or more pull sutures.

Example 117. The prosthetic heart valve of any example herein, particularly example 116, wherein the second outer skirt is configured to detach from the second portion of the plurality of interconnected struts and enable separation of the annular frame from the second outer skirt via removal of the one or more pull sutures from respective the suture lines and withdrawal of the stitches through the second outer skirt.

Example 118. The prosthetic heart valve of any example herein, particularly any of examples 112-115, wherein the second outer skirt is secured to the second portion of the plurality of interconnected struts with bioabsorbable sutures, the bioabsorbable sutures configured to dissolve to enable separation of the annular frame from the second outer skirt.

Example 119. The prosthetic heart valve of any example herein, particularly any of examples 112 or 113, wherein the first outer skirt is secured to the first portion of the plurality of interconnected struts with bioabsorbable sutures, the bioabsorbable sutures configured to dissolve to enable separation of the annular frame from the first outer skirt.

Example 120. The prosthetic heart valve of any example herein, particularly any of examples 112-119, wherein the first outer skirt and/or the second outer skirt is made of pericardium.

Example 121. A prosthetic heart valve comprising: an annular frame comprising an annulus portion and an aortic portion; and a first outer skirt detachably secured to the outer surface of the aortic portion.

Example 122. The prosthetic heart valve of any example herein, particularly example 121, wherein the first outer skirt is detachably secured to the outer surface of the aortic portion with bioabsorbable sutures.

Example 123. The prosthetic heart valve of any example herein, particularly any of examples 121-122, wherein the first outer skirt is detachably secured to a plurality of interconnected struts of the aortic portion with at least one retaining suture and one or more pull sutures.

Example 124. The prosthetic heart valve of any example herein, particularly any of examples 121-123, wherein the first outer skirt is made of pericardium.

Example 125. The prosthetic heart valve of any example herein, particularly any of examples 121-124, further comprising a second outer skirt secured to the outer surface of the annulus portion.

Example 126. The prosthetic heart valve of any example herein, particularly example 125, wherein the second outer skirt is detachably secured to the outer surface of the annulus portion with bioabsorbable sutures.

Example 127. The prosthetic heart valve of any example herein, particularly example 125, wherein the second outer skirt is detachably secured to a plurality of interconnected struts of the annulus portion with at least one retaining suture and one or more pull sutures.

Example 128. The prosthetic heart valve of any example herein, particularly any of examples 125-127, wherein the second outer skirt is made of pericardium.

Example 129. A prosthetic heart valve of any example herein, particularly any of examples 1-128, wherein the prosthetic heart valve is sterilized.

The features described herein with regard to any example can be combined with other features described in any one or more of the other examples, unless otherwise stated. For example, any one or more of the features of one prosthetic valve can be combined with any one or more features of another prosthetic valve. As another example, any one or more features of one outer skirt can be combined with any one or more features of another outer skirt. As yet another example, any one or more features of one method can be combined with any one or more features of another method.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. A prosthetic heart valve comprising:
an annular frame comprising a plurality of interconnected struts; and
an outer skirt disposed on an outer surface of the annular frame and detachably secured to at least a portion of the plurality of interconnected struts;
wherein the outer skirt is detachably secured to the portion of the plurality of interconnected struts via at least one retaining suture and one or more pull sutures, the retaining suture and one or more pull sutures arranged along a suture line, the retaining suture forming a plurality of stitches extending through the outer skirt and over the one or more pull sutures; and
wherein the outer skirt is configured to detach from the portion of the plurality of interconnected struts and enable separation of the annular frame from the outer skirt via removal of the one or more pull sutures from the suture line and withdrawal of the plurality of stitches through the outer skirt.

2. The prosthetic heart valve of claim 1, wherein each of the one or more pull sutures comprises an end region that extends beyond the suture line, the end region being attached to an attachment region at one or more of the annular frame, the outer skirt, or a valvular structure disposed within an interior of the annular frame; and
wherein the end region is configured to be released from the attachment region and pulled to remove a corresponding pull suture of the one or more pull sutures from the suture line.

3. The prosthetic heart valve of claim 2, wherein the end region is releasably attached at the attachment region via one or more of an adhesive, a suture, a knot, or an elastic band.

4. The prosthetic heart valve of claim 2, wherein the end region comprises a marker configured to enable identification of a corresponding pull suture of the one or more pull sutures.

5. The prosthetic heart valve of claim 4, wherein the marker comprises at least one of a colored marker or a radiopaque marker attached to or disposed within the end region.

6. The prosthetic heart valve of claim 1, wherein the one or more pull sutures are comprised of one or more of PTFE, ePTFE, or prolene.

7. The prosthetic heart valve of claim 1, wherein the at least one retaining suture comprises UHMPE force fiber.

8. The prosthetic heart valve of claim 1, wherein the at least one retaining suture extends through pre-formed openings in the outer skirt.

9. The prosthetic heart valve of claim 1, wherein each stitch extends through a single opening in the outer skirt.

10. The prosthetic heart valve of claim 1, wherein the outer skirt comprises two or more skirt portions, adjacent ones of the two or more skirt portions having an overlapping region therebetween, the suture line extending through at least a portion of the overlapping region.

11. The prosthetic heart valve of claim 1, wherein the outer skirt comprises a first outer skirt disposed on an inflow end portion of the outer surface of the annular frame and detachably secured to a first portion of the plurality of interconnected struts forming the inflow end portion of the annular frame, and wherein the prosthetic heart valve further comprises a second outer skirt disposed on an outflow end portion of the outer surface of the annular frame and detachably secured to a second portion of the plurality of interconnected struts forming the outflow end portion of the annular frame.

12. The prosthetic heart valve of claim 1, wherein the plurality of stitches further extend around the portion of the plurality of interconnected struts.

13. The prosthetic heart valve of claim 12, wherein the plurality of stitches are configured to remain attached to the portion of the plurality of interconnected struts after separation of the annular frame from the outer skirt during an explant procedure.

14. The prosthetic heart valve of claim 1, wherein the outer skirt is further detachably secured to the portion of the plurality of interconnected struts via a plurality of non-penetrating stitches, the plurality of non-penetrating stitches extending around the portion of the plurality of interconnected struts and between the at least one retaining suture and an interior surface of the outer skirt.

15. The prosthetic heart valve of claim 14, wherein the plurality of stitches and the plurality of non-penetrating stitches are configured to remain attached to the portion of the plurality of interconnected struts after separation of the annular frame from the outer skirt during an explant procedure.

16. The prosthetic heart valve of claim 1, wherein the outer skirt comprises at least one fused edge, and wherein the fused edge is folded over an exterior surface of the prosthetic heart valve to create a folded edge, wherein the folded edge is secured to adjacent struts of the plurality of interconnected struts via the at least one retaining suture and the one or more pull sutures.

17. A prosthetic heart valve comprising:
an annular frame comprising a plurality of interconnected struts; and
an outer skirt disposed on an outer surface of the annular frame and detachably secured to at least a portion of the plurality of interconnected struts;
wherein the outer skirt is detachably secured to the portion of the plurality of interconnected struts via at least one retaining suture and one or more pull sutures, the retaining suture and one or more pull sutures arranged along a suture line, the retaining suture forming a plurality of stitches extending through the outer skirt and over the one or more pull sutures.

18. A prosthetic heart valve comprising:
an annular frame comprising a plurality of interconnected struts and a plurality of anchoring projections; and
an outer skirt disposed on an outer surface of the annular frame and covering at least a portion of the plurality of anchoring projections, the outer skirt detachably secured to at least a portion of the plurality of interconnected struts;
wherein the outer skirt is configured to detach from the portion of the plurality of interconnected struts and enable separation of the annular frame from the outer skirt, the plurality of anchoring projections on the outer surface of the annular frame being exposed after separation of the annular frame and the outer skirt.

19. The prosthetic heart valve of claim 18, wherein the outer skirt is detachably secured to the portion of the plurality of interconnected struts via at least one retaining suture and one or more pull sutures, the retaining suture and one or more pull sutures arranged along a suture line, the retaining suture forming a plurality of stitches extending around the portion of the plurality of interconnected struts, through the outer skirt, and over the one or more pull sutures, and wherein the outer skirt can be detached from the annular frame via removal of the one or more pull sutures from the suture line and withdrawal of the plurality of stitches through the outer skirt.

20. The prosthetic heart valve of claim 19, wherein each of the one or more pull sutures comprises an end region that extends beyond the suture line, the end region being releasably attached to an attachment region at one or more of the annular frame, the outer skirt, or a valvular structure disposed within an interior of the annular frame; and
wherein the end region is configured to be released from the attachment region and pulled to remove a corresponding pull suture of the one or more pull sutures from the suture line.
